SEQUENCE LISTING (92 sequences)

SEQ ID NO: 1 - DNA, 774 bp, Homo sapiens
agggcaagttaagggaatagtggaatgaaggttcatttttcattctcacaaactaatgaaaccctgcttatcttaaaccaacctgctcactggagcagggaggacaggaccagcataaaaggcagggcagagtcgactgttgcttacactttcttctgacataacagtgttcactagcaacctcaaacagacaccatggtgcatctgactcctgaggagaagactgctgtcaatgccctgtggggcaaagtgaacgtggatgcagttggtggtgaggccctgggcagattactggtggtctacccttggacccagaggttctttgagtcctttggggatctgtcctctcctgatgctgttatgggcaaccctaaggtgaaggctcatggcaagaaggtgctaggtgcctttagtgatggcctggctcacctggacaacctcaagggcactttttctcagctgagtgagctgcactgtgacaagctgcacgtggatcctgagaacttcaggctcttgggcaatgtgctggtgtgtgtgctggcccgcaactttggcaaggaattcaccccacaaatgcaggctgcctatcagaaggtggtggctggtgtggctaatgccctggctcacaagtaccattgagatcctggactgtttcctgataaccataagaagaccctatttccctagattctattttctgaacttgggaacacaatgcctacttcaagggtatggcttctgcctaataaagaatgttcagctcaacttcctgat

SEQ ID NO: 2 - DNA, 4953 bp, Homo sapiens
gaacgagtgggaacgtagctggtcgcagagggcaccagcggctgcaggacttcaccaagggaccctgaggctcgtgagcagggacccgcggtgcgggttatgctgggggctcagatcaccgtagacaactggacactcaggaccacgccatggaggagctgcaggatgattatgaagacatgatggaggagaatctggagcaggaggaatatgaagacccagacatccccgagtcccagatggaggagccggcagctcacgacaccgaggcaacagccacagactaccacaccacatcacacccgggtacccacaaggtctatgtggagctgcaggagctggtgatggacgaaaagaaccaggagctgagatggatggaggcggcgcgctgggtgcaactggaggagaacctgggggagaatggggcctggggccgcccgcacctctctcacctcaccttctggagcctcctagagctgcgtagagtcttcaccaagggtactgtcctcctagacctgcaagagacctccctggctggagtggccaaccaactgctagacaggtttatctttgaagaccagatccggcctcaggaccgagaggagctgctccgggccctgctgcttaaacacagccacgctggagagctggaggccctggggggtgtgaagcctgcagtcctgacacgctctggggatccttcacagcctctgctcccccaacactcctcactggagacacagctcttctgtgagcagggagatgggggcacagaagggcactcaccatctggaattctggaaaagattcccccggattcagaggccacgttggtgctagtgggccgcgccgacttcctggagcagccggtgctgggcttcgtgaggctgcaggaggcagcggagctggaggcggtggagctgccggtgcctatacgcttcctctttgtgttgctgggacctgaggccccccacatcgattacacccagcttggccgggctgctgccaccctcatgtcagagagggtgttccgcatagatgcctacatggctcagagccgaggggagctgctgcactccctagagggcttcctggactgcagcctagtgctgcctcccaccgatgccccctccgagcaggcactgctcagtctggtgcctgtgcagagggagctacttcgaaggcgctatcagtccagccctgccaagccagactccagcttctacaagggcctagacttaaatgggggcccagatgaccctctgcagcagacaggccagctcttcgggggcctggtgcgtgatatccggcgccgctacccctattacctgagtgacatcacagatgcattcagcccccaggtcctggctgccgtcatcttcatctactttgctgcactgtcacccgccatcaccttcggcggcctcctgggagaaaagacccggaaccagatgggagtgtcggagctgctgatctccactgcagtgcagggcattctcttcgccctgctgggggctcagcccctgcttgtggtcggcttctcaggacccctgctggtgtttgaggaagccttcttctcgttctgcgagaccaacggtctagagtacatcgtgggccgcgtgtggatcggcttctggctcatcctgctggtggtgttggtggtggccttcgagggtagcttcctggtccgcttcatctcccgctatacccaggagatcttctccttcctcatttccctcatcttcatctatgagactttctccaagctgatcaagatcttccaggaccacccactacagaagacttataactacaacgtgttgatggtgcccaaacctcagggccccctgcccaacacagccctcctctcccttgtgctcatggccggtaccttcttctttgccatgatgctgcgcaagttcaagaacagctcctatttccctggcaagctgcgtcgggtcatcggggacttcggggtccccatctccatcctgatcatggtcctggtggatttcttcattcaggatacctacacccagaaactctcggtgcctgatggcttcaaggtgtccaactcctcagcccggggctgggtcatccacccactgggcttgcgttccgagtttcccatctggatgatgtttgcctccgccctgcctgctctgctggtcttcatcctcatattcctggagtctcagatcaccacgctgattgtcagcaaacctgagcgcaagatggtcaagggctccggcttccacctggacctgctgctggtagtaggcatgggtggggtggccgccctctttgggatgccctggctcagtgccaccaccgtgcgttccgtcacccatgccaacgccctcactgtcatgggcaaagccagcaccccaggggctgcagcccagatccaggaggtcaaagagcagcggatcagtggactcctggtcgctgtgcttgtgggcctgtccatcctcatggagcccatcctgtcccgcatccccctggctgtactgtttggcatcttcctctacatgggggtcacgtcgctcagcggcatccagctctttgaccgcatcttgcttctgttcaagccacccaagtatcacccagatgtgccctacgtcaagcgggtgaagacctggcgcatgcacttattcacgggcatccagatcatctgcctggcagtgctgtgggtggtgaagtccacgccggcctccctggccctgcccttcgtcctcatcctcactgtgccgctgcggcgcgtcctgctgccgctcatcttcaggaacgtggagcttcagtgtctggatgctgatgatgccaaggcaacctttgatgaggaggaaggtcgggatgaatacgacgaagtggccatgcctgtgtgaggggcgggcccaggccctagaccctcccccaccattccacatccccaccttccaaggaaaagcagaagttcatgggcacctcatggactccaggatcctcctggagcagcagctgaggccccagggctgtgggtggggaaggaaggcgtgtccaggagaccttccacaaagggtagcctggcttttctggctggggatggccgatggggcccacattagggggtttgttgcacagtccctcctgttgccacactttcactggggatcccgtgctggaagacttagatctgagccctccctcttcccagcacaggcaggggtagaagcaaaggcaggaggtgggtgagcgggtggggtgcttgctgtgtgaccttgggcaagtcccttgacctttccagcctatatttcctcttctgtaaaatgggtatattgatgataatacccacattacaggatggttactgaggaccaaagatacatgtaaaatagggctttgtaaactccacagggactgttctatagcagtcatcatttgtctttgaacgtacccaaggtcacatagctgggatttgaactgagccgtgcagctgggatttgaaccaggccttctgatttcaaggtccgagctctgtcctctgtcagtcatgcgtccactttcccttcccctgtgactcctcccttccccactctgctcccagcccctaccttgagaccctcttctctgggcccagagagaggcgtcctggtgaggacaaggtacaggcaaggatgatccagggattgggcctgggactcaggcctcctaagtgtttggttcctccctccaaacactcattagttcactcattcattcattccacaaacatttactgagggccccggaatcagtggactccgaggggactgagacaagccctgccctggggtgggggtggggggcaaggtacagttgattctacatttggatagggagtgggggagggtgggaaggtaggggcgggagagtgagggggtttgtaatttattaattgcgtattttctaagagttttcaacatagtttggcttcacacacaacttcaggcccctcatttgagagccattatcctcaactccatctaaactgaatcttggggagaacccagatctgaccaattggggtaggagacagcaggctctccaagaacatgggcaaatttatttttttataaaacaaaaagataaaaagagttgaaagacgtgaaagtggtgagagatggaggaaacagaatcaggaagtggtagaaaagagaggaggtggctgggcgcagtggctcacgtttgtaatcccagcactttgggaggccaagttgggcggatcatttgaggtcaggagtttgagaccagcctggccaacatggtgaaaccccgttactactaaaaatacaaaaattagctgggtgtctcgtggcaggcacctgtaatcccagctacttagaaggctgaggcaagagaatcacctgaacccaggaggtggaggttgcagtgagccaagattgcaccactgcactccagcctgggcaacagagcgagaccctgtctcaaaaaaaaaaaaaaaaaaaaaaaaaaaacggaaggaaacatcagccttgggggccacagactcaacatgtgtgtgtggtggggttccagcccaacatagagtaacattatttgtacctcccaggctagctcagtccatgggaggctctcctgtccctgaaagctgacacccacctttcaccacttcgcccatgctacagttcagtttcctcgtctgtaaaatggggatgataatggtacctaccttgcagtgttgttataaggattaaaggagacagtgcaagaaaaggccttggttggtgaagagcccaacctcggaggggagctgctgggatcctccttatcttgactgggatgtccctgtctccccctccccttgctccttgaacatggccaaggaaagtgaaaaacaaaaattattcactctgctagcacccttccccttgatgcctgggaataggttttgccaataaacgtatctgtgttgga

SEQ ID NO: 3 - DNA, 601 bp, Homo sapiens
gggagatttcaacgtgtttaaatacatcagccatctaggaaaggacatctcttgagacttcacttcagcttcactgacttctggattctcctcttgagtaaaaggactcagccaactatgaagttttttgtttttgctttaatcttggctctcatgctttccatgactggagctgattcacatgcaaagagacatcatgggtataaaagaaaattccatgaaaagcatcattcacatcgaggctatagatcaaattatctgtatgacaattgatatcttcagtaatcacggggcatgattatggaggtttgactggcaaattcgctttggactcgtgtattctcatttgtcataccgcatcacactaccactgctttttgaagaattatcataaggcaatgcagaataaaagaaataccatgatttagtgaattctgtgtttcaggatacttcccttcctaattatcatttgattagatacttgcaatttaaatgttaagctgttttcactgctgtttctgagtaatagaaattcattcctctccaaaagcaataaaattcaagcacattattatgtgaaaaaaaaaaaaaaaaaaaaaaa

SEQ ID NO: 4 - DNA, 2276 bp, Homo sapiens
aagcccagcagccccggggcggatggctccggccgcctggctccgcagcgcggccgcgcgcgccctcctgcccccgatgctgctgctgctgctccagccgccgccgctgctggcccgggctctgccgccggacgcccaccacctccatgccgagaggagggggccacagccctggcatgcagccctgcccagtagcccggcacctgcccctgccacgcaggaagccccccggcctgccagcagcctcaggcctccccgctgtggcgtgcccgacccatctgatgggctgagtgcccgcaaccgacagaagaggttcgtgctttctggcgggcgctgggagaagacggacctcacctacaggatccttcggttcccatggcagttggtgcaggagcaggtgcggcagacgatggcagaggccctaaaggtatggagcgatgtgacgccactcacctttactgaggtgcacgagggccgtgctgacatcatgatcgacttcgccaggtactggcatggggacgacctgccgtttgatgggcctgggggcatcctggcccatgccttcttccccaagactcaccgagaaggggatgtccacttcgactatgatgagacctggactatcggggatgaccagggcacagacctgctgcaggtggcagcccatgaatttggccacgtgctggggctgcagcacacaacagcagccaaggccctgatgtccgccttctacacctttcgctacccactgagtctcagcccagatgactgcaggggcgttcaacacctatatggccagccctggcccactgtcacctccaggaccccagccctgggcccccaggctgggatagacaccaatgagattgcaccgctggagccagacgccccgccagatgcctgtgaggcctcctttgacgcggtctccaccatccgaggcgagctctttttcttcaaagcgggctttgtgtggcgcctccgtgggggccagctgcagcccggctacccagcattggcctctcgccactggcagggactgcccagccctgtggacgctgccttcgaggatgcccagggccacatttggttcttccaaggtgctcagtactgggtgtacgacggtgaaaagccagtcctgggccccgcacccctcaccgagctgggcctggtgaggttcccggtccatgctgccttggtctggggtcccgagaagaacaagatctacttcttccgaggcagggactactggcgtttccaccccagcacccggcgtgtagacagtcccgtgccccgcagggccactgactggagaggggtgccctctgagatcgacgctgccttccaggatgctgatggctatgcctacttcctgcgcggccgcctctactggaagtttgaccctgtgaaggtgaaggctctggaaggcttcccccgtctcgtgggtcctgacttctttggctgtgccgagcctgccaacactttcctctgaccatggcttggatgccctcaggggtgctgacccctgccaggccacgaatatcaggctagagacccatggccatctttgtggctgtgggcaccaggcatgggactgagcccatgtctcctcagggggatggggtggggtacaaccaccatgacaactgccgggagggccacgcaggtcgtggtcacctgccagcgactgtctcagactgggcagggaggctttggcatgacttaagaggaagggcagtcttgggcccgctatgcaggtcctggcaaacctggctgccctgtctccatccctgtccctcagggtagcaccatggcaggactgggggaactggagtgtccttgctgtatccctgttgtgaggttccttccaggggctggcactgaagcaagggtgctggggccccatggccttcagccctggctgagcaactgggctgtagggcagggccacttcctgaggtcaggtcttggtaggtgcctgcatctgtctgccttctggctgacaatcctggaaatctgttctccagaatccaggccaaaaagttcacagtcaaatggggaggggtattcttcatgcaggagaccccaggccctggaggctgcaacatacctcaatcctgtcccaggccggatcctcctgaagcccttttcgcagcactgctatcctccaaagccattgtaaatgtgtgtacagtgtgtataaaccttcttcttctttttttttttttaaactgaggattgtcattaaacacagttgttttctaaaaaaaaaaaaaaaa

SEQ ID NO: 5 - DNA, 2715 bp, Homo sapiens
gcttcgcagcgtcacgccctccggggccgtggcggcgacggcggtgcgtagcttactcacaggggcggcccgtatccctccgccgccggcgcggctcggccctccctcccctggcccgccaatccccgcgcctcccgacctgcccctcggtcgggcccaccccgtgctccgacggccccaccccggcggcgcagcccgcccgcccgcgcgtccctcggtccacctgcagcagggaggaagacaggcaatccctccggctgtccgaccaagagaggccggccgagcccgaggcttgggcttttgctttctggcggagggatctgcggcggtttaggaggcggcgctgatcctgggaggaagaggcagctacggcggcggcggcggtggcggctagggcggcggcgaataaaggggccgccgccgggtgatgcggtgaccgctgcggcaggcccaggagctgagtgggccccggccctcagcccgtcccgccggacccgctttcctcaactctccatcttctcctgccgaccgagatcgccgaggcggcctcaggctccctagccccttccccgtcccttccccgcccccgtccccgccccgggggccgccgccacccgcctcccaccatggctctgaagagaatccacaaggaattgaatgatctggcacgggaccctccagcacagtgttcagcaggtcctgttggagatgatatgttccattggcaagctacaataatggggccaaatgacagtccctatcagggtggagtatttttcttgacaattcatttcccaacagattaccccttcaaaccacctaaggttgcatttacaacaagaatttatcatccaaatattaacagtaatggcagcatttgtcttgatattctacgatcacagtggtctccagcactaactatttcaaaagtactcttgtccatctgttctctgttgtgtgatcccaatccagatgatcctttagtgcctgagattgctcggatctacaaaacagatagagaaaagtacaacagaatagctcgggaatggactcagaagtatgcgatgtaattaaagaaattattggataacctctacaaataaagataggggaactctgaaagagaaagtccttttgatttccatttgactgctttctatgagcccacgcctcatcttcccctgtgcacatgtttacctgatacagcagtgctgcgtgttgtacatacttggaacaacaaactagaaatactgtacttctgtaccaacattgcctcctagcagagaagtgtgtgtgtgacaagccagttctacaggcattacctaggtgtgagactaaaagcttttcttattgacttaaatttggataacagcaaggtgtgaggggggtggtgggtatggtgtgtgcttggatgggaaagaaaaggctccactcacctataggagattatttttaagtggaatccatttaaactcaaaacagttatgaaaagcaaggtgaagaacatgaagctgtgtctgtattcattttattccgaaggagctacgtcttaggtgaaagttatgaccaaccagattaaactctacccacatcctgcattttaaggtctaagtttaactggtcaacatttaaatggattggagctattagtacatcaagtgtgatgggctttgttcccaactcttttacatctccctaccccttcaacctttggcctttcagcccttctttctctcttccatattctttggtttgtatgtggtttctcagttaatacatagctaatagctcttatttttcttatgtttttaaccgcttaggtctatttggatgtaagggtgaaaattcatttgatggaaatacttgtgtatatttaaagacccaattgctcctctggagcttgtactttcaagaatgattaatctgtgtaataaactggttactacagtcattacatataattttgtgtgaataggctttttcatttttaagaagtttgtctagctgagattagtggtggattttctcccacttctgaaatgttcatttatactggttgcattttaagatcatgaaacaattccagttacattgtaaaaaggatatcttacgagtaattttattgaacaagttagaggcataagcttaagagcatttccatgaaacaacacatgcagcattccaggaacttgattgttaaattcaataagaaatttgctttattaatgaaactaagctgcatttcatcaaaaccttgtgacattcccttggtacataggacataaaacacagaggcattgctatttggtaagttaagcttctgtgattgtaattataaaagagcaacattgaccaaacctgggaaacaagagcacagtcttgtttggagagtctacataattactttgcactaacatttgcaggatgttcacacaattttaaattgtactgtatgtggctttttgaagtcttcccttgaccctagtaaaatatagcttgaaacttgtaaacaactgtgtttgccagaaacatcattcatgtgaactaggcaagttaccttttttccccccttcttttcctaattgtaaactaggccaacctgaaagccatggctgatgctctagccatcaggttctttcaaatgcatctttacactcttgcacaaaagttaaggaataaatgtccactgcttttggttttaaaaaaaaaaaaaaa

SEQ ID NO: 6 - DNA, 105 bp, Homo sapiens
ccgcaactttggcaaggaattcaccccacaaatgcaggctgcctatcagaaggtggtggctggtgtggctaatgccctggctcacaagtaccattgagatcctgg

SEQ ID NO: 7 - DNA, 120 bp, Homo sapiens
tgatggaggagaatctggagcaggaggaatatgaagacccagacatccccgagtcccagatggaggagccggcagctcacgacaccgaggcaacagccacagactaccacaccacatcac

SEQ ID NO: 8 - DNA, 114 bp, Homo sapiens
ttcacatcgaggctatagatcaaattatctgtatgacaattgatatcttcagtaatcacggggcatgattatggaggtttgactggcaaattcgctttggactcgtgtattctc

SEQ ID NO: 9 - DNA, 168 bp, Homo sapiens
aggtggcagcccatgaatttggccacgtgctggggctgcagcacacaacagcagccaaggccctgatgtccgccttctacacctttcgctacccactgagtctcagcccagatgactgcaggggcgttcaacacctatatggccagccctggcccactgtcacctcca

SEQ ID NO: 10 - DNA, 170 bp, Homo sapiens
gccaaatgacagtccctatcagggtggagtatttttcttgacaattcatttcccaacagattaccccttcaaaccacctaaggttgcatttacaacaagaatttatcatccaaatattaacagtaatggcagcatttgtcttgatattctacgatcacagtggtctccag

SEQ ID NO: 11 - DNA, 19 bp, Homo sapiens
ccgcaactttggcaaggaa

SEQ ID NO: 12 - DNA, 24 bp, Homo sapiens
ccaggatctcaatggtacttgtga

SEQ ID NO: 13 - DNA, 21 bp, Homo sapiens
tgatggaggagaatctggagc

SEQ ID NO: 14 - DNA, 22 bp, Homo sapiens
gtgatgtggtgtggtagtctgt

SEQ ID NO: 15 - DNA, 21 bp, Homo sapiens
ttcacatcgaggctatagatc

SEQ ID NO: 16 - DNA, 21 bp, Homo sapiens
gagaatacacgagtccaaagc

SEQ ID NO: 17 - DNA, 19 bp, Homo sapiens
aggtggcagcccatgaatt

SEQ ID NO: 18 - DNA, 19 bp, Homo sapiens
ggtcctggaggtgacagtg

SEQ ID NO: 19 - DNA, 23 bp, Homo sapiens
gccaaatgacagtccctatcagg

SEQ ID NO: 20 - DNA, 25 bp, Homo sapiens
gcactaaaggatcatctggattggg

SEQ ID NO: 21 - DNA, 1793 bp, Homo sapiens
cgcgtccgccccgcgagcacagagcctcgcctttgccgatccgccgcccgtccacacccgccgccagctcaccatggatgatgatatcgccgcgctcgtcgtcgacaacggctccggcatgtgcaaggccggcttcgcgggcgacgatgccccccgggccgtcttcccctccatcgtggggcgccccaggcaccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagcgggaaatcgtgcgtgacattaaggagaagctgtgctacgtcgccctggacttcgagcaagagatggccacggctgcttccagctcctccctggagaagagctacgagctgcctgacggccaggtcatcaccattggcaatgagcggttccgctgccctgaggcactcttccagccttccttcctgggcatggagtcctgtggcatccacgaaactaccttcaactccatcatgaagtgtgacgtggacatccgcaaagacctgtacgccaacacagtgctgtctggcggcaccaccatgtaccctggcattgccgacaggatgcagaaggagatcactgccctggcacccagcacaatgaagatcaagatcattgctcctcctgagcgcaagtactccgtgtggatcggcggctccatcctggcctcgctgtccaccttccagcagatgtggatcagcaagcaggagtatgacgagtccggcccctccatcgtccaccgcaaatgcttctaggcggactatgacttagttgcgttacaccctttcttgacaaaacctaacttgcgcagaaaacaagatgagattggcatggctttatttgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcattgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttccccctttttgtcccccaacttgagatgtatgaaggcttttggtctccctgggagtgggtggaggcagccagggcttacctgtacactgacttgagaccagttgaataaaagtgcacacctta

SEQ ID NO: 22 - DNA, 1718 bp, Homo sapiens
gagggtgcataagttctctagtagggtgatgatataaaaagccaccggagcactccataaggcacaaactttcagagacagcagagcacacaagcttctaggacaagagccaggaagaaaccaccggaaggaaccatctcactgtgtgtaaacatgacttccaagctggccgtggctctcttggcagccttcctgatttctgcagctctgtgtgaaggtgcagttttgccaaggagtgctaaagaacttagatgtcagtgcataaagacatactccaaacctttccaccccaaatttatcaaagaactgagagtgattgagagtggaccacactgcgccaacacagaaattattgtaaagctttctgatggaagagagctctgtctggaccccaaggaaaactgggtgcagagggttgtggagaagtttttgaagagggctgagaattcataaaaaaattcattctctgtggtatccaagaatcagtgaagatgccagtgaaacttcaagcaaatctacttcaacacttcatgtattgtgtgggtctgttgtagggttgccagatgcaatacaagattcctggttaaatttgaatttcagtaaacaatgaatagtttttcattgtaccatgaaatatccagaacatacttatatgtaaagtattatttatttgaatctacaaaaaacaacaaataatttttaaatataaggattttcctagatattgcacgggagaatatacaaatagcaaaattgaggccaagggccaagagaatatccgaactttaatttcaggaattgaatgggtttgctagaatgtgatatttgaagcatcacataaaaatgatgggacaataaattttgccataaagtcaaatttagctggaaatcctggatttttttctgttaaatctggcaaccctagtctgctagccaggatccacaagtccttgttccactgtgccttggtttctcctttatttctaagtggaaaaagtattagccaccatcttacctcacagtgatgttgtgaggacatgtggaagcactttaagttttttcatcataacataaattattttcaagtgtaacttattaacctatttattatttatgtatttatttaagcatcaaatatttgtgcaagaatttggaaaaatagaagatgaatcattgattgaatagttataaagatgttatagtaaatttattttattttagatattaaatgatgttttattagataaatttcaatcagggtttttagattaaacaaacaaacaattgggtacccagttaaattttcatttcagataaacaacaaataattttttagtataagtacattattgtttatctgaaattttaattgaactaacaatcctagtttgatactcccagtcttgtcattgccagctgtgttggtagtgctgtgttgaattacggaataatgagttagaactattaaaacagccaaaactccacagtcaatattagtaatttcttgctggttgaaacttgtttattatgtacaaatagattcttataatattatttaaatgactgcatttttaaatacaaggctttatatttttaactttaagatgtttttatgtgctctccaaattttttttactgtttctgattgtatggaaatataaaagtaaatatgaaacatttaaaatataatttgttgtcaaagtaaaaaaaaaaaaaaa

SEQ ID NO: 23 - DNA, 573 bp, Homo sapiens
ctccattccattatacctttgagtatataaaacagctacaatattccagggccagtcacttgccatttctcataacagcgtcagagagaaagaactgactgaaacgtttgagatgaagaaagttctcctcctgatcacagccatcttggcagtggctgttggtttcccagtctctcaagaccaggaacgagaaaaaagaagtatcagtgacagcgatgaattagcttcagggttttttgtgttcccttacccatatccatttcgcccacttccaccaattccatttccaagatttccatggtttagacgtaattttcctattccaatacctgaatctgcccctacaactccccttcctagcgaaaagtaaacaagaaggaaaagtcacgataaacctggtcacctgaaattgaaattgagccacttccttgaagaatcaaaattcctgttaataaaagaaaaacaaatgtaattgaaatagcacacagcattctctagtcaatatctttagtgatcttctttaataaacttgaaagcaaagattttggtttcttaatttccacaaaaaaaaaa

SEQ ID NO: 24 - DNA, 2406 bp, Homo sapiens
agaggcaggggctggcctgggatgcgcgcgcacctgccctcgccccgccccgcccgcacgaggggtggtggccgaggccccgccccgcacgcctcgcctgaggcgggtccgctcagcccaggcgcccgcccccgcccccgccgattaaatgggccggcggggctcagcccccggaaacggtcgtacacttcggggctgcgagcgcggagggcgacgacgacgaagcgcagacagcgtcatggcagagcaggtggccctgagccggacccaggtgtgcgggatcctgcgggaagagcttttccagggcgatgccttccatcagtcggatacacacatattcatcatcatgggtgcatcgggtgacctggccaagaagaagatctaccccaccatctggtggctgttccgggatggccttctgcccgaaaacaccttcatcgtgggctatgcccgttcccgcctcacagtggctgacatccgcaaacagagtgagcccttcttcaaggccaccccagaggagaagctcaagctggaggacttctttgcccgcaactcctatgtggctggccagtacgatgatgcagcctcctaccagcgcctcaacagccacatgaatgccctccacctggggtcacaggccaaccgcctcttctacctggccttgcccccgaccgtctacgaggccgtcaccaagaacattcacgagtcctgcatgagccagataggctggaaccgcatcatcgtggagaagcccttcgggagggacctgcagagctctgaccggctgtccaaccacatctcctccctgttccgtgaggaccagatctaccgcatcgaccactacctgggcaaggagatggtgcagaacctcatggtgctgagatttgccaacaggatcttcggccccatctggaaccgggacaacatcgcctgcgttatcctcaccttcaaggagccctttggcactgagggtcgcgggggctatttcgatgaatttgggatcatccgggacgtgatgcagaaccacctactgcagatgctgtgtctggtggccatggagaagcccgcctccaccaactcagatgacgtccgtgatgagaaggtcaaggtgttgaaatgcatctcagaggtgcaggccaacaatgtggtcctgggccagtacgtggggaaccccgatggagagggcgaggccaccaaagggtacctggacgaccccacggtgccccgcgggtccaccaccgccacttttgcagccgtcgtcctctatgtggagaatgagaggtgggatggggtgcccttcatcctgcgctgcggcaaggccctgaacgagcgcaaggccgaggtgaggctgcagttccatgatgtggccggcgacatcttccaccagcagtgcaagcgcaacgagctggtgatccgcgtgcagcccaacgaggccgtgtacaccaagatgatgaccaagaagccgggcatgttcttcaaccccgaggagtcggagctggacctgacctacggcaacagatacaagaacgtgaagctccctgacgcctatgagcgcctcatcctggacgtcttctgcgggagccagatgcacttcgtgcgcagcgacgagctccgtgaggcctggcgtattttcaccccactgctgcaccagattgagctggagaagcccaagcccatcccctatatttatggcagccgaggccccacggaggcagacgagctgatgaagagagtgggtttccagtatgagggcacctacaagtgggtgaacccccacaagctctgagccctgggcacccacctccacccccgccacggccaccctccttcccgccgcccgaccccgagtcgggaggactccgggaccattgacctcagctgcacattcctggccccgggctctggccaccctggcccgcccctcgctgctgctactacccgagcccagctacattcctcagctgccaagcactcgagaccatcctggcccctccagaccctgcctgagcccaggagctgagtcacctcctccactcactccagcccaacagaaggaaggaggagggcgcccattcgtctgtcccagagcttattggccactgggtctcactcctgagtggggccagggtgggagggagggacgagggggaggaaaggggcgagcacccacgtgagagaatctgcctgtggccttgcccgccagcctcagtgccacttgacattccttgtcaccagcaacatctcgagccccctggatgtcccctgtcccaccaactctgcactccatggccaccccgtgccacccgtaggcagcctctctgctataagaaaagcagacgcagcagctgggacccctcccaacctcaatgccctgccattaaatccgcaaacagcccaaaaaaaaaa

SEQ ID NO: 25 - DNA, 1310 bp, Homo sapiens
aaattgagcccgcagcctcccgcttcgctctctgctcctcctgttcgacagtcagccgcatcttcttttgcgtcgccagccgagccacatcgctcagacaccatggggaaggtgaaggtcggagtcaacggatttggtcgtattgggcgcctggtcaccagggctgcttttaactctggtaaagtggatattgttgccatcaatgaccccttcattgacctcaactacatggtttacatgttccaatatgattccacccatggcaaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgatgcccccatgttcgtcatgggtgtgaaccatgagaagtatgacaacagcctcaagatcatcagcaatgcctcctgcaccaccaactgcttagcacccctggccaaggtcatccatgacaactttggtatcgtggaaggactcatgaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgacaacgaatttggctacagcaacagggtggtggacctcatggcccacatggcctccaaggagtaagacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaacc

SEQ ID NO: 26 - DNA, 2660 bp, Homo sapiens
gcaggaaggtgggcctggaagataacagctagcaggctaaggtcagacactgacacttgcagttgtctttggtagtttttttgcactaacttcaggaaccagctcatgatctcaggatgtatggaaaaataatctttgtattactattgtcagaaattgtgagcatatcagcattaagtaccactgaggtggcaatgcacacttcaacttcttcttcagtcacaaagagttacatctcatcacagacaaatgatacgcacaaacgggacacatatgcagccactcctagagctcatgaagtttcagaaatttctgttagaactgtttaccctccagaagaggaaaccggagaaagggtacaacttgcccatcatttctctgaaccagagataacactcattatttttggggtgatggctggtgttattggaacgatcctcttaatttcttacggtattcgccgactgataaagaaaagcccatctgatgtaaaacctctcccctcacctgacacagacgtgcctttaagttctgttgaaatagaaaatccagagacaagtgatcaatgagaatctgttcaccaaaccaaatgtggaaagaacacaaagaagacataagacttcagtcaagtgaaaaattaacatgtggactggacactccaataaattatatacctgcctaagttgtacaatttcagaatgcaattttcattataatgagttccagtgactcaatgatggggaaaaaaatctctgctcattaatatttcaagataaagaacaaatgtttccttgaatgcttgcttttgtgtgttagcataatttttagaattgtttgagaattctgatccaaaactttagttgaattcatctacgtttgtttaatattaacttaacctattctattgtattataatgatgattctgtcaaatgaaaggcttgaaatacctagatgaagtttagattttcttcctattgtaaacttttgagtctggtttcattgttttaaataaattaaggggacactaaagtcctatcattcatttccttcattgctgaacaggcaagatataatattacatgaatgattactatattttgttcacactaataaagcttatgctcagaaatgccatacacacacacacacacacacacaaacacacacatttatcatttaatgcataaatcaacacaaaaggttttcccattaatatgaaatattacatatatataagtgccatatttaaaataatttgtctaacagtagaactgtgtcggagcactcactgaagcttgcattccactgaaagagttatttgtgtaagtagagtatccggagaaggaaaagaacttacgacctttctttataacagaaactcaactctaaattcaacaagatgtgcaaaccggacatgcaggtgaatattttaataggttactataaggttctcaattaaattctttaatctgtccagtcccagtttctcttattaataaaactttggaaattgctttaaaccatttaaaggaaatttctagatatagaaactaaggactgtgactatacagctgtcactcatttgtagtaaaacttaaaaagcaaaaacaaaaaacaaaaaagaccttcctgtgatactttatttccgaactaataaaaatctatatgactttttattattgtgtgataaccaagtaaatgttttctattttgcatattttcaggcatggtaacagaaatttaccttttaataaattaaaaaatctaaattttaacctacttgtatgttcggagagtgtttttgtactatattgactacttaaaatagagaatgagactaagaagggaacatttctgttgatacatgttttttaaaagtaattttaagagcattattaggttaattaatccaattaatgacccaaatgccaaggtaattttaaatttacatttttaataaaagcaacatgttgaaacaagagagggtgagattaacctttttgctaaagtaatttacaagtcaaagacaggaagagatcagagtgaatgtgccttcttaaccagagctacagaatttagtgaataattaaagtacaaactgctttgacctccttgaacttttccaagcaatttctctgtacttctatatatgaatgtcttagccaattttctgctactataacagaatacgacagactgggtaatttaaaaagaaaagaaatttattttcttcctagttctggaggctgggaaggcgaagggcatggcactgacatctgccttgtaactgatgagaaccttcttactgcatgataacaaagcagcaaggcaagcaaaagcgtaagatgaagagagaggaaatgaagccaaacacatcctttcatcagaagcccattccctctataaggcgttattacatttatgagaatggagtcctcatgacctaatcgtgaccttaaaggcccctcccaacactgttacaatggcaattaaatttcaacaaaggttccagaggtgacattcgaatcagcaatgaaattttcatagttaaatttggtattcgtgggggaagaaatgaccatttcccttgtatttttataattaaatcagcaaaatattgtaataaagaaatctttcctgtgaagataccatgacccca

SEQ ID NO: 27 - DNA, 626 bp, Homo sapiens
acatttgcttctgacacaactgtgttcactagcaacctcaaacagacaccatggtgcatctgactcctgaggagaagtctgccgttactgccctgtggggcaaggtgaacgtggatgaagttggtggtgaggccctgggcaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcaggctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgc

SEQ ID NO: 28 - DNA, 1743 bp, Homo sapiens
aaagaaggtaagggcagtgagaatgatgcatcttgcattccttgtgctgttgtgtctgccagtctgctctgcctatcctctgagtggggcagcaaaagaggaggactccaacaaggatcttgcccagcaatacctagaaaagtactacaacctcgaaaaggatgtgaaacagtttagaagaaaggacagtaatctcattgttaaaaaaatccaaggaatgcagaagttccttgggttggaggtgacagggaagctagacactgacactctggaggtgatgcgcaagcccaggtgtggagttcctgacgttggtcacttcagctcctttcctggcatgccgaagtggaggaaaacccaccttacatacaggattgtgaattatacaccagatttgccaagagatgctgttgattctgccattgagaaagctctgaaagtctgggaagaggtgactccactcacattctccaggctgtatgaaggagaggctgatataatgatctctttcgcagttaaagaacatggagacttttactcttttgatggcccaggacacagtttggctcatgcctacccacctggacctgggctttatggagatattcactttgatgatgatgaaaaatggacagaagatgcatcaggcaccaatttattcctcgttgctgctcatgaacttggccactccctggggctctttcactcagccaacactgaagctttgatgtacccactctacaactcattcacagagctcgcccagttccgcctttcgcaagatgatgtgaatggcattcagtctctctacggacctccccctgcctctactgaggaacccctggtgcccacaaaatctgttccttcgggatctgagatgccagccaagtgtgatcctgctttgtccttcgatgccatcagcactctgaggggagaatatctgttctttaaagacagatatttttggcgaagatcccactggaaccctgaacctgaatttcatttgatttctgcattttggccctctcttccatcatatttggatgctgcatatgaagttaacagcagggacaccgtttttatttttaaaggaaatgagttctgggccatcagaggaaatgaggtacaagcaggttatccaagaggcatccataccctgggttttcctccaaccataaggaaaattgatgcagctgtttctgacaaggaaaagaagaaaacatacttctttgcagcggacaaatactggagatttgatgaaaatagccagtccatggagcaaggcttccctagactaatagctgatgactttccaggagttgagcctaaggttgatgctgtattacaggcatttggatttttctacttcttcagtggatcatcacagtttgagtttgaccccaatgccaggatggtgacacacatattaaagagtaacagctggttacattgctaggcgagatagggggaagacagatatgggtgtttttaataaatctaataattattcatctaatgtattatgagccaaaatggttaatttttcctgcatgttctgtgactgaagaagatgagccttgcagatatctgcatgtgtcatgaagaatgtttctggaattcttcacttgcttttgaattgcactgaacagaattaagaaatactcatgtgcaataggtgagagaatgtattttcatagatgtgttattacttcctcaataaaaagttttattttgggcctgttcctt

SEQ ID NO: 29 - DNA, 2276 bp, Homo sapiens; identical to SEQ ID NO: 4

SEQ ID NO: 30 - DNA, 1828 bp, Homo sapiens
ctacaaggaggcaggcaagacagcaaggcatagagacaacatagagctaagtaaagccagtggaaatgaagagtcttccaatcctactgttgctgtgcgtggcagtttgctcagcctatccattggatggagctgcaaggggtgaggacaccagcatgaaccttgttcagaaatatctagaaaactactacgacctcaaaaaagatgtgaaacagtttgttaggagaaaggacagtggtcctgttgttaaaaaaatccgagaaatgcagaagttccttggattggaggtgacggggaagctggactccgacactctggaggtgatgcgcaagcccaggtgtggagttcctgatgttggtcacttcagaacctttcctggcatcccgaagtggaggaaaacccaccttacatacaggattgtgaattatacaccagatttgccaaaagatgctgttgattctgctgttgagaaagctctgaaagtctgggaagaggtgactccactcacattctccaggctgtatgaaggagaggctgatataatgatctcttttgcagttagagaacatggagacttttacccttttgatggacctggaaatgttttggcccatgcctatgcccctgggccagggattaatggagatgcccactttgatgatgatgaacaatggacaaaggatacaacagggaccaatttatttctcgttgctgctcatgaaattggccactccctgggtctctttcactcagccaacactgaagctttgatgtacccactctatcactcactcacagacctgactcggttccgcctgtctcaagatgatataaatggcattcagtccctctatggacctccccctgactcccctgagacccccctggtacccacggaacctgtccctccagaacctgggacgccagccaactgtgatcctgctttgtcctttgatgctgtcagcactctgaggggagaaatcctgatctttaaagacaggcacttttggcgcaaatccctcaggaagcttgaacctgaattgcatttgatctcttcattttggccatctcttccttcaggcgtggatgccgcatatgaagttactagcaaggacctcgttttcatttttaaaggaaatcaattctgggctatcagaggaaatgaggtacgagctggatacccaagaggcatccacaccctaggtttccctccaaccgtgaggaaaatcgatgcagccatttctgataaggaaaagaacaaaacatatttctttgtagaggacaaatactggagatttgatgagaagagaaattccatggagccaggctttcccaagcaaatagctgaagactttccagggattgactcaaagattgatgctgtttttgaagaatttgggttcttttatttctttactggatcttcacagttggagtttgacccaaatgcaaagaaagtgacacacactttgaagagtaacagctggcttaattgttgaaagagatatgtagaaggcacaatatgggcactttaaatgaagctaataattcttcacctaagtctctgtgaattgaaatgttcgttttctcctgcctgtgctgtgactcgagtcacactcaagggaacttgagcgtgaatctgtatcttgccggtcatttttatgttattacagggcattcaaatgggctgctgcttagcttgcaccttgtcacatagagtgatctttcccaagagaaggggaagcactcgtgtgcaacagacaagtgactgtatctgtgtagactatttgcttatttaataaagacgatttgtcagttattttatctt

SEQ ID NO: 31 - DNA, 1147 bp, Homo sapiens
accaaatcaaccataggtccaagaacaattgtctctggacggcagctatgcgactcaccgtgctgtgtgctgtgtgcctgctgcctggcagcctggccctgccgctgcctcaggaggcgggaggcatgagtgagctacagtgggaacaggctcaggactatctcaagagattttatctctatgactcagaaacaaaaaatgccaacagtttagaagccaaactcaaggagatgcaaaaattctttggcctacctataactggaatgttaaactcccgcgtcatagaaataatgcagaagcccagatgtggagtgccagatgttgcagaatactcactatttccaaatagcccaaaatggacttccaaagtggtcacctacaggatcgtatcatatactcgagacttaccgcatattacagtggatcgattagtgtcaaaggctttaaacatgtggggcaaagagatccccctgcatttcaggaaagttgtatggggaactgctgacatcatgattggctttgcgcgaggagctcatggggactcctacccatttgatgggccaggaaacacgctggctcatgcctttgcgcctgggacaggtctcggaggagatgctcacttcgatgaggatgaacgctggacggatggtagcagtctagggattaacttcctgtatgctgcaactcatgaacttggccattctttgggtatgggacattcctctgatcctaatgcagtgatgtatccaacctatggaaatggagatccccaaaattttaaactttcccaggatgatattaaaggcattcagaaactatatggaaagagaagtaattcaagaaagaaatagaaacttcaggcagaacatccattcattcattcattggattgtatatcattgttgcacaatcagaattgataagcactgttcctccactccatttagcaattatgtcacccttttttattgcagttggtttttgaatgtctttcactccttttaaggataaactcctttatggtgtgactgtgtcttattcatctatacttgcagtgggtagatgtcaataaatgttacatacacaaataaataaaatgtttattccatggtaaatttaaaaaaaaaaaaaaaaaaaaaaaaaaaaa

SEQ ID NO: 32 - DNA, 1307 bp, Homo sapiens
acttatctgcagacttgtaggcagcaactcaccctcactcagaggtcttctggttctggaaacaactctagctcagccttctccaccatgagcctcagacttgataccaccccttcctgtaacagtgcgagaccacttcatgccttgcaggtgctgctgcttctgtcattgctgctgactgctctggcttcctccaccaaaggacaaactaagagaaacttggcgaaaggcaaagaggaaagtctagacagtgacttgtatgctgaactccgctgcatgtgtataaagacaacctctggaattcatcccaaaaacatccaaagtttggaagtgatcgggaaaggaacccattgcaaccaagtcgaagtgatagccacactgaaggatgggaggaaaatctgcctggacccagatgctcccagaatcaagaaaattgtacagaaaaaattggcaggtgatgaatctgctgattaatttgttctgtttctgccaaacttctttaactcccaggaagggtagaattttgaaaccttgattttctagagttctcatttattcaggatacctattcttactgtattaaaatttggatatgtgtttcattctgtctcaaaaatcacattttattctgagaaggttggttaaaagatggcagaaagaagatgaaaataaataagcctggtttcaaccctctaattcttgcctaaacattggactgtactttgcatttttttctttaaaaatttctattctaacacaacttggttgatttttcctggtctactttatggttattagacatactcatgggtattattagatttcataatggtcaatgataataggaattacatggagcccaacagagaatatttgctcaatacatttttgttaatatatttaggaacttaatggagtctctcagtgtcttagtcctaggatgtcttatttaaaatactccctgaaagtttattctgatgtttattttagccatcaaacactaaaataataaattggtgaatatgaatcttataaactgtggttagctggtttaaagtgaatatatttgccactagtagaacaaaaatagatgatgaaaatgaattaacatatctacatagttataattctatcattagaatgagccttataaataagtacaatataggacttcaaccttactagactcctaattctaaattctacttttttcatcaacagaactttcattcattttttaaaccctaaaacttatacccacactattcttacaaaaatattcacatgaaataaaaatttgctattga

SEQ ID NO: 33 - DNA, 740 bp, Homo sapiens
gcacagagttgggagtgactccagagcctccagcgagatgctgctgattctgctgtcagtggccctgctggccctgagctcagctgagagttcaagtgaagatgtcagccaggaagaatctctcttcctaatatcaggaaagccagaaggacgacgcccacaaggaggaaaccagccccaacgtcccccacctcctccaggaaagccacaaggaccacccccacaaggaggaaaccagtcccaaggtcccccacctcctccaggaaagccagaaggacgacccccacaaggaggcaaccagtcccaaggtcccccacctcatccaggaaagccagaaagaccacccccacaaggaggaaaccagtcccaaggaaagccacaaggaccaccccaacaagaaggcaacaagcctcaaggtcccccacctcctggaaagccacaaggcccacccccagcaggaggcaatccccagcagcctcaggcacctcctgctggaaagccccaggggccacctccacctcctcaagggggcaggccacccagacctgcccagggacaacagcctccccagtaatctaggattcaatgacaggaagtgaataagaagatatcagtgaattcaaataattcaattgctacaaatgccgtgacattggaacaaggtcatcatagctctaactttaatataccaataaaataatcagcttgcaaaaaaaaaaaaaaaaaaaaaaaaaaaaaaa

SEQ ID NO: 34 - DNA, 4953 bp, Homo sapiens; identical to SEQ ID NO: 2

SEQ ID NO: 35 - DNA, 578 bp, Homo sapiens
gagtgtttaaatacattggccctctagggtagcacatcatctcttgaagcttcacttcaacttcactacttctgtagtctcatcttgagtaaaagagaacccagccaactatgaagttccttgtctttgccttcatcttggctctcatggtttccatgattggagctgattcatctgaagagtatgggtatggcccttatcagccagttccagaacaaccactatacccacaaccataccaaccacaataccaacaatataccttttaatatcatcagtaactgcaggacatgattattgaggcttgattggcaaatacgacttctacatccatattctcatctttcataccatatcacactactaccactttttgaagaatcatcaaagagcaatgcaaatgaaaaacactataatttactgtatactctttgtttcaggatacttgccttttcaattgtcacttgatgatataattgcaatttaaactgttaagctgtgttcagtactgtttctgaataatagaaatcacttctctaaaagcaataaatttcaagcacatttttacataaaaaaaa

SEQ ID NO: 36 - DNA, 3897 bp, Homo sapiens
cagtttgcaaaagccagaggtgcaagaagcagcgactgcagcagcagcagcagcagcggcggtggcagcagcagcagcagcggcggcagcagcagcagcagcggaggcaccggtggcagcagcagcatcaccagcaacaacaacaaaaaaaaatcctcatcaaatcctcacctaagctttcagtgtatccagatccacatcttcactcaagccaggagagggaaagaggaaaggggggcaggaaaaaaaaaaaacccaacaacttagcggaaacttctcagagaatgctccaaaactcagcagtgcttctggtgctggtgatcagtgcttctgcaacccatgaggcggagcagaatgactctgtgagccccaggaaatcccgagtggcggctcaaaactcagctgaagtggttcgttgcctcaacagtgctctacaggtcggctgcggggcttttgcatgcctggaaaactccacctgtgacacagatgggatgtatgacatctgtaaatccttcttgtacagcgctgctaaatttgacactcagggaaaagcattcgtcaaagagagcttaaaatgcatcgccaacggggtcacctccaaggtcttcctcgccattcggaggtgctccactttccaaaggatgattgctgaggtgcaggaagagtgctacagcaagctgaatgtgtgcagcatcgccaagcggaaccctgaagccatcactgaggtcgtccagctgcccaatcacttctccaacagatactataacagacttgtccgaagcctgctggaatgtgatgaagacacagtcagcacaatcagagacagcctgatggagaaaattgggcctaacatggccagcctcttccacatcctgcagacagaccactgtgcccaaacacacccacgagctgacttcaacaggagacgcaccaatgagccgcagaagctgaaagtcctcctcaggaacctccgaggtgaggaggactctccctcccacatcaaacgcacatcccatgagagtgcataaccagggagaggttattcacaacctcaccaaactagtatcattttaggggtgttgacacaccagttttgagtgtactgtgcctggtttgatttttttaaagtagttcctattttctatcccccttaaagaaaattgcatgaaactaggcttctgtaatcaatatcccaacattctgcaatggcagcattcccaccaacaaaatccatgtgaccattctgcctctcctcaggagaaagtaccctcttttaccaacttcctctgccatgtttttcccctgctcccctgagaccacccccaaacacaaaacattcatgtaactctccagccattgtaatttgaagatgtggatccctttagaacggttgccccagtagagttagctgataaggaaactttatttaaatgcatgtcttaaatgctcataaagatgttaaatggaattcgtgttatgaatctgtgctggccatggacgaatatgaatgtcacatttgaattcttgatctctaatgagctagtgtcttatggtcttgatcctccaatgtctaattttctttccgacacatttaccaaattgcttgagcctggctgtccaaccagactttgagcctgcatcttcttgcatctaatgaaaaacaaaaagctaacatctttacgtactgtaactgctcagagctttaaaagtatctttaacaattgtcttaaaaccagagaatcttaaggtctaactgtggaatataaatagctgaaaactaatgtactgtacataaattccagaggactctgcttaaacaaagcagtatataataactttattgcatatagatttagttttgtaacttagctttatttttcttttcctgggaatggaataactatctcacttccagatatccacataaatgctccttgtggccttttttataactaagggggtagaagtagttttaattcaacatcaaaacttaagatgggcctgtatgagacaggaaaaaccaacaggtttatctgaaggaccccaggtaagatgttaatctcccagcccacctcaacccagaggctactcttgacttagacctatactgaaagatctctgtcacatccaactggaaattccaggaaccaaaaagagcatccctatgggcttggaccacttacagtgtgataaggcctactatacattaggaagtggcagttctttactcgtcccctttcatcggtgcctggtactctggcaaatgatgatggggtgggagactttccattaaatcaatcaggaatgagtcaatcagcctttaggtctttagtccgggggacttggggctgagagagtataaataaccctgggctgtccagccttaatagacttctcttacattttcgtcctgtagcacgctgcctgccaaagtagtcctggcagctggaccatctctgtaggatcgtaaaaaaatagaaaaaaagaaaaaaaaaagaaagaaagagggaaaaagagctggtggtttgatcatttctgccatgatgtttacaagatggcgaccaccaaagtcaaacgactaacctatctatgaacaacagtagtttctcagggtcactgtccttgaacccaacagtcccttatgagcgtcactgcccaccaaaggtcaatgtcaagagaggaagagagggaggaggggtaggactgcaggggccactccaaactcgcttaggtagaaactattggtgcttgactctcactaggctaaactcaagatttgaccaaatcgagtgatagggatcctggtgggaggagagagggcacatctccagaaaaatgaaaagcaatacaactttaccataaagcctttaaaaccagtaacgtgctgctcaaggaccaagagcaattgcagcagacccagcagcagcagcagcagcacaaacattgctgcctttgtccccacacagcctctaagcgtgctgacatcagattgttaagggcatttttatactcagaactgtcccatccccaggtccccaaacttatggacactgccttagcctcttggaaatcaggtagaccatattctaagttagactcttcccctccctcccacacttcccacccccaggcaaggctgacttctctgaatcagaaaagctattaaagtttgtgtgttgtgtccattttgcaaacccaactaagccaggaccccaatgcgacaagtagttcatgagtattcctagcaaatttctctctttcttcagttcagtagatttccttttttcttttctttttttttttttttttttttggctgtgacctcttcaaaccgtggtacccccccttttctccccacgatgatatctatatatgtatctacaatacatatatctacacatacagaaagaagcagttctcacaatgttgctagttttttgcttctctttcccccaccctactccctccaattcccccttaaacttccaaagcttcgtcttgtgtttgctgcagagtgattcgggggctgacctagaccagtttgcatgattcttctcttgtgatttggttgcactttagacatttttgtgccattatatttgcattatgtatttataatttaaatgatatttaggtttttggctgagtactggaataaacagtgagcatatctggtatatgtcattatttattgttaaattacatttttaagctccatgtgcatataaaggttatgaaacatatcatggtaatgacagatgcaagttattttatttgcttatttttataattaaagatgccatagcataatatgaagcctttggtgaattccttctaagataaaaataataataaagtgttacgttttattggtttcaaaaaaaaaaaaaaaaaaaa

SEQ ID NO: 37 - DNA, 2804 bp, Homo sapiens
gaccctctagccggaagccacgcctgcccactagcccgacgcccgcctggcgggaacatgggctcgcccctcaccagcgatctgcagtcagttggtagcgcctgcacgtcgcgcgcggtgttcgattgtcgctgcctggggaggaggagccggagccgccgccgccgccgccgccgccgcgggcttcgttcgtaaggaagggggcctaggcccgggcctgcggtggtgggggttgctgcgcgccgggggtcgctcctgctgtgtcttccgctccagcttcgcccacttccccttgccagcggggtgggcgcggagaagacctgccggagccatggaggacgaagtggtccgctttgccaagaagatggacaagatggtgcagaagaagaacgcggctggagcattggatttgctaaaggagcttaagaatattcctatgaccctggaattactgcagtccacaagaatcggaatgtcagttaatgctattcgcaagcagagtacagatgaggaagttacatctttggcaaagtctctcatcaaatcctggaaaaaattattagatgggccatcaactgagaaagaccttgacgaaaagaagaaagaacctgcaattacatcgcagaacagccctgaggcaagagaagaaagtacttccagcggcaatgtaagcaacagaaaggatgagacaaatgctcgagatacttatgtttcatcctttcctcgggcaccaagcacttctgattctgtgcggttgaagtgtagggagatgcttgctgcagctcttcgaacaggggatgactacattgcaattggagctgatgaggaagaattaggatctcaaattgaagaagctatatatcaagaaataaggaatacagacatgaaatacaaaaatagagtacgaagtaggatatcaaatcttaaagatgcaaaaaatccaaatttaaggaaaaatgtcctctgtgggaatattcctcctgacttatttgctagaatgacagcagaggaaatggctagtgatgagctgaaagagatgcggaaaaacttgaccaaagaagccatcagagagcatcagatggccaagactggtgggacccagactgacttgttcacatgtggcaaatgtaaaaagaagaattgcacttacacacaggtacaaacccgtagtgctgatgaaccaatgacaacatttgttgtctgtaatgaatgtggaaatcgatggaagttctgttgagttggaagaattggcaaaatatctggaccattaagaaaacggattttgtaactagctttaaactaggccaagcaactagttttcctgcaaatcaaatttttaaagcaacttgggttagactttgtttttgacctaacatcccttccttaaatgccttctgtagtttcagatcagtagggagaccatataataattttatggtacctgtttcaaaacatattttttctgtttttataagtaagttgatattaattaaactcttggcaatatttcttctttcttaaaggaaaatataccttaactttttttcttttacactgtgaaacatacacagtagaaattctgttactctctgttattaatacataaatgaaaatacatttttttccatattggcatgtagctacaaatattaaaggaggagaaaaggtaatataattttaggtttaccaaatatggtgtgtattcaaataatacttgaccagcttatctaaaatgtacataattttgaggtagcttatgaatttgattttaattattatgttcacaagcttggaatattagatattattttgcatctgtaactaaccgtgatcatcatttcttgtaatttcttgtacatgtatattacttgttcttaatagatttttggaaacaagactttattgagatcagtttggttttcctgttaatttacctgtttgactttataatgtgttttagttttgcagaagaacactgttgtagtttagaaggcttttcataaatcccctcataggcaaagatgaaaacttcccactatttttttcccctcttaggaagacatactggaaagaaaatgtttagcatcttagtgtagtatagctattgtaaacagttcatgactagattttgattcggaaatctatactgaccaaggattaatcttaaggattgtataattcattaaagctgtggtctttccatgtggagactgatagaaaataattttgtcccaagtcttatttgctgactttttctgtcatgagtgagattgttgaacaaactgaatatatgggctatagcaagtagctttacagtacagatcttacaattaagttttgcttttgttaaagtgtgtaccattttttctgtttggagtaagacaaaaattgttttgacataggttccctagggtacacttgctctagcatactttaaaggccactgttgcaaagtctacattttatgctgaatctgcattctgtcaggcacccgtagaaagacctcagtacatgctttgcactctcctttgctccctttttccaatttcttattgcatatcattttgttgtaatacagaaagcagcatttttaaatgtccgtgttaagaattggcccactggtaccaactcacctctattttgtcagttcatagttgaagattttgttttatttcaaaaacaaagtacatttttgaaataatgtttcagaataaaataatctcacttttaagtgatccattttaaaatttgtaattcaataaagttttttttgttgttaaacatataaaaaaaaaaaaaaaaaa

SEQ ID NO: 38 - DNA, 2879 bp, Homo sapiens
gcttcgcagcgtcacgccctccggggccgtggcggcgacggcggtgcgtagcttactcacaggggcggcccgtatccctccgccgccggcgcggctcggccctccctcccctggcccgccaatccccgcgcctcccgacctgcccctcggtcgggcccaccccgtgctccgacggccccaccccggcggcgcagcccgcccgcccgcgcgtccctcggtccacctgcagcagggaggaagacaggcaatccctccggctgtccgaccaagagaggccggccgagcccgaggcttgggcttttgctttctggcggagggatctgcggcggtttaggaggcggcgctgatcctgggaggaagaggcagctacggcggcggcggcggtggcggctagggcggcggcgaataaaggggccgccgccgggtgatgcggtgaccgctgcggcaggcccaggagctgagtgggccccggccctcagcccgtcccgccggacccgctttcctcaactctccatcttctcctgccgaccgagatcgccgaggcggcctcaggctccctagccccttccccgtcccttccccgcccccgtccccgccccgggggccgccgccacccgcctcccaccatggctctgaagagaatccacaagctccctccacaaaaccgcctgagctcgggctgacagaggaagccgttttgcccgatccacaagtatatcctgagttcacttacctcttgggtggcagcacacatcggtccaccctgcttgtccagaaactgttaagagttggaagttcagaagaaaaaaaaaaggaattgaatgatctggcacgggaccctccagcacagtgttcagcaggtcctgttggagatgatatgttccattggcaagctacaataatggggccaaatgacagtccctatcagggtggagtatttttcttgacaattcatttcccaacagattaccccttcaaaccacctaaggttgcatttacaacaagaatttatcatccaaatattaacagtaatggcagcatttgtcttgatattctacgatcacagtggtctccagcactaactatttcaaaagtactcttgtccatctgttctctgttgtgtgatcccaatccagatgatcctttagtgcctgagattgctcggatctacaaaacagatagagaaaagtacaacagaatagctcgggaatggactcagaagtatgcgatgtaattaaagaaattattggataacctctacaaataaagataggggaactctgaaagagaaagtccttttgatttccatttgactgctttctatgagcccacgcctcatcttcccctgtgcacatgtttacctgatacagcagtgctgcgtgttgtacatacttggaacaacaaactagaaatactgtacttctgtaccaacattgcctcctagcagagaagtgtgtgtgtgacaagccagttctacaggcattacctaggtgtgagactaaaagcttttcttattgacttaaatttggataacagcaaggtgtgaggggggtggtgggtatggtgtgtgcttggatgggaaagaaaaggctccactcacctataggagattatttttaagtggaatccatttaaactcaaaacagttatgaaaagcaaggtgaagaacatgaagctgtgtctgtattcattttattccgaaggagctacgtcttaggtgaaagttatgaccaaccagattaaactctacccacatcctgcattttaaggtctaagtttaactggtcaacatttaaatggattggagctattagtacatcaagtgtgatgggctttgttcccaactcttttacatctccctaccccttcaacctttggcctttcagcccttctttctctcttccatattctttggtttgtatgtggtttctcagttaatacatagctaatagctcttatttttcttatgtttttaaccgcttaggtctatttggatgtaagggtgaaaattcatttgatggaaatacttgtgtatatttaaagacccaattgctcctctggagcttgtactttcaagaatgattaatctgtgtaataaactggttactacagtcattacatataattttgtgtgaataggctttttcatttttaagaagtttgtctagctgagattagtggtggattttctcccacttctgaaatgttcatttatactggttgcattttaagatcatgaaacaattccagttacattgtaaaaaggatatcttacgagtaattttattgaacaagttagaggcataagcttaagagcatttccatgaaacaacacatgcagcattccaggaacttgattgttaaattcaataagaaatttgctttattaatgaaactaagctgcatttcatcaaaaccttgtgacattcccttggtacataggacataaaacacagaggcattgctatttggtaagttaagcttctgtgattgtaattataaaagagcaacattgaccaaacctgggaaacaagagcacagtcttgtttggagagtctacataattactttgcactaacatttgcaggatgttcacacaattttaaattgtactgtatgtggctttttgaagtcttcccttgaccctagtaaaatatagcttgaaacttgtaaacaactgtgtttgccagaaacatcattcatgtgaactaggcaagttaccttttttccccccttcttttcctaattgtaaactaggccaacctgaaagccatggctgatgctctagccatcaggttctttcaaatgcatctttacactcttgcacaaaagttaaggaataaatgtccactgcttttggttttaaaaaaaaaaaaaaa

SEQ ID NO: 39 - DNA, 100 bp, Homo sapiens
tgcagaaggagatcactgccctggcacccagcacaatgaagatcaagatcattgctcctcctgagcgcaagtactccgtgtggatcggcggctccatcct

SEQ ID NO: 40 - DNA, 100 bp, Homo sapiens
agttttgccaaggagtgctaaagaacttagatgtcagtgcataaagacatactccaaacctttccaccccaaatttatcaaagaactgagagtgattgag

SEQ ID NO: 41 - DNA, 100 bp, Homo sapiens
cagccatcttggcagtggctgttggtttcccagtctctcaagaccaggaacgagaaaaaagaagtatcagtgacagcgatgaattagcttcagggttttt

SEQ ID NO: 42 - DNA, 100 bp, Homo sapiens
gcccatcccctatatttatggcagccgaggccccacggaggcagacgagctgatgaagagagtgggtttccagtatgagggcacctacaagtgggtgaac

SEQ ID NO: 43 - DNA, 100 bp, Homo sapiens
cactcctccacctttgacgctggggctggcattgccctcaacgaccactttgtcaagctcatttcctggtatgacaacgaatttggctacagcaacaggg

SEQ ID NO: 44 - DNA, 100 bp, Homo sapiens
tctgttagaactgtttaccctccagaagaggaaaccggagaaagggtacaacttgcccatcatttctctgaaccagagataacactcattatttttgggg

SEQ ID NO: 45 - DNA, 100 bp, Homo sapiens
tgtccactcctgatgctgttatgggcaaccctaaggtgaaggctcatggcaagaaagtgctcggtgcctttagtgatggcctggctcacctggacaacct

SEQ ID NO: 46 - DNA, 100 bp, Homo sapiens
gcagcaaaagaggaggactccaacaaggatcttgcccagcaatacctagaaaagtactacaacctcgaaaaggatgtgaaacagtttagaagaaaggaca

SEQ ID NO: 47 - DNA, 100 bp, Homo sapiens
agcagccaaggccctgatgtccgccttctacacctttcgctacccactgagtctcagcccagatgactgcaggggcgttcaacacctatatggccagccc

SEQ ID NO: 48 - DNA, 100 bp, Homo sapiens
tggacctggaaatgttttggcccatgcctatgcccctgggccagggattaatggagatgcccactttgatgatgatgaacaatggacaaaggatacaaca

SEQ ID NO: 49 - DNA, 100 bp, Homo sapiens
gatgggccaggaaacacgctggctcatgcctttgcgcctgggacaggtctcggaggagatgctcacttcgatgaggatgaacgctggacggatggtagca

SEQ ID NO: 50 - DNA, 100 bp, Homo sapiens
actccgctgcatgtgtataaagacaacctctggaattcatcccaaaaacatccaaagtttggaagtgatcgggaaaggaacccattgcaaccaagtcgaa

SEQ ID NO: 51 - DNA, 100 bp, Homo sapiens
agggacaacagcctccccagtaatctaggattcaatgacaggaagtgaataagaagatatcagtgaattcaaataattcaattgctacaaatgccgtgac

SEQ ID NO: 52 - DNA, 100 bp, Homo sapiens
ttgctgcactgtcacccgccatcaccttcggcggcctcctgggagaaaagacccggaaccagatgggagtgtcggagctgctgatctccactgcagtgca

SEQ ID NO: 53 - DNA, 100 bp, Homo sapiens
actatacccacaaccataccaaccacaataccaacaatataccttttaatatcatcagtaactgcaggacatgattattgaggcttgattggcaaatacg

SEQ ID NO: 54 - DNA, 100 bp, Homo sapiens
taacagacttgtccgaagcctgctggaatgtgatgaagacacagtcagcacaatcagagacagcctgatggagaaaattgggcctaacatggccagcctc

SEQ ID NO: 55 - DNA, 100 bp, Homo sapiens
aggagcttaagaatattcctatgaccctggaattactgcagtccacaagaatcggaatgtcagttaatgctattcgcaagcagagtacagatgaggaagt

SEQ ID NO: 56 - DNA, 100 bp, Homo sapiens
caaaagtactcttgtccatctgttctctgttgtgtgatcccaatccagatgatcctttagtgcctgagattgctcggatctacaaaacagatagagaaaa

SEQ ID NO: 57 - DNA, 50 bp, Homo sapiens
tgcagaaggagatcactgccctggcacccagcacaatgaagatcaagatc

SEQ ID NO: 58 - DNA, 50 bp, Homo sapiens
attgctcctcctgagcgcaagtactccgtgtggatcggcggctccatcct

SEQ ID NO: 59 - DNA, 50 bp, Homo sapiens
agttttgccaaggagtgctaaagaacttagatgtcagtgcataaagacat

SEQ ID NO: 60 - DNA, 50 bp, Homo sapiens
actccaaacctttccaccccaaatttatcaaagaactgagagtgattgag

SEQ ID NO: 61 - DNA, 50 bp, Homo sapiens
cagccatcttggcagtggctgttggtttcccagtctctcaagaccaggaa

SEQ ID NO: 62 - DNA, 50 bp, Homo sapiens
cgagaaaaaagaagtatcagtgacagcgatgaattagcttcagggttttt

SEQ ID NO: 63 - DNA, 50 bp, Homo sapiens
gcccatcccctatatttatggcagccgaggccccacggaggcagacgagc

SEQ ID NO: 64 - DNA, 50 bp, Homo sapiens
tgatgaagagagtgggtttccagtatgagggcacctacaagtgggtgaac

SEQ ID NO: 65 - DNA, 50 bp, Homo sapiens
cactcctccacctttgacgctggggctggcattgccctcaacgaccactt

SEQ ID NO: 66 - DNA, 50 bp, Homo sapiens
tgtcaagctcatttcctggtatgacaacgaatttggctacagcaacaggg

SEQ ID NO: 67 - DNA, 50 bp, Homo sapiens
tctgttagaactgtttaccctccagaagaggaaaccggagaaagggtaca

SEQ ID NO: 68 - DNA, 50 bp, Homo sapiens
acttgcccatcatttctctgaaccagagataacactcattatttttgggg

SEQ ID NO: 69 - DNA, 50 bp, Homo sapiens
tgtccactcctgatgctgttatgggcaaccctaaggtgaaggctcatggc

SEQ ID NO: 70 - DNA, 50 bp, Homo sapiens
aagaaagtgctcggtgcctttagtgatggcctggctcacctggacaacct

SEQ ID NO: 71 - DNA, 50 bp, Homo sapiens
gcagcaaaagaggaggactccaacaaggatcttgcccagcaatacctaga

SEQ ID NO: 72 - DNA, 50 bp, Homo sapiens
aaagtactacaacctcgaaaaggatgtgaaacagtttagaagaaaggaca

SEQ ID NO: 73 - DNA, 50 bp, Homo sapiens
agcagccaaggccctgatgtccgccttctacacctttcgctacccactga

SEQ ID NO: 74 - DNA, 50 bp, Homo sapiens
gtctcagcccagatgactgcaggggcgttcaacacctatatggccagccc

SEQ ID NO: 75 - DNA, 50 bp, Homo sapiens
tggacctggaaatgttttggcccatgcctatgcccctgggccagggatta

SEQ ID NO: 76 - DNA, 50 bp, Homo sapiens
atggagatgcccactttgatgatgatgaacaatggacaaaggatacaaca

SEQ ID NO: 77 - DNA, 50 bp, Homo sapiens
gatgggccaggaaacacgctggctcatgcctttgcgcctgggacaggtct

SEQ ID NO: 78 - DNA, 50 bp, Homo sapiens
cggaggagatgctcacttcgatgaggatgaacgctggacggatggtagca

SEQ ID NO: 79 - DNA, 50 bp, Homo sapiens
actccgctgcatgtgtataaagacaacctctggaattcatcccaaaaaca

SEQ ID NO: 80 - DNA, 50 bp, Homo sapiens
tccaaagtttggaagtgatcgggaaaggaacccattgcaaccaagtcgaa

SEQ ID NO: 81 - DNA, 50 bp, Homo sapiens
agggacaacagcctccccagtaatctaggattcaatgacaggaagtgaat

SEQ ID NO: 82 - DNA, 50 bp, Homo sapiens
aagaagatatcagtgaattcaaataattcaattgctacaaatgccgtgac

SEQ ID NO: 83 - DNA, 50 bp, Homo sapiens
ttgctgcactgtcacccgccatcaccttcggcggcctcctgggagaaaag

SEQ ID NO: 84 - DNA, 50 bp, Homo sapiens
acccggaaccagatgggagtgtcggagctgctgatctccactgcagtgca

SEQ ID NO: 85 - DNA, 50 bp, Homo sapiens
actatacccacaaccataccaaccacaataccaacaatataccttttaat

SEQ ID NO: 86 - DNA, 50 bp, Homo sapiens
atcatcagtaactgcaggacatgattattgaggcttgattggcaaatacg

SEQ ID NO: 87 - DNA, 50 bp, Homo sapiens
taacagacttgtccgaagcctgctggaatgtgatgaagacacagtcagca

SEQ ID NO: 88 - DNA, 50 bp, Homo sapiens
caatcagagacagcctgatggagaaaattgggcctaacatggccagcctc

SEQ ID NO: 89 - DNA, 50 bp, Homo sapiens
aggagcttaagaatattcctatgaccctggaattactgcagtccacaaga

SEQ ID NO: 90 - DNA, 50 bp, Homo sapiens
atcggaatgtcagttaatgctattcgcaagcagagtacagatgaggaagt

SEQ ID NO: 91 - DNA, 50 bp, Homo sapiens
caaaagtactcttgtccatctgttctctgttgtgtgatcccaatccagat

SEQ ID NO: 92 - DNA, 50 bp, Homo sapiens
gatcctttagtgcctgagattgctcggatctacaaaacagatagagaaaa

(19) United States
(12) Patent Application Publication
(10) Pub. No.: US 2018/0371523 A1
(43) Pub. Date: Dec. 27, 2018

(54) METHODS AND MATERIALS FOR DETECTING RNA SEQUENCES

(71) Applicant: INSTITUTE OF ENVIRONMENTAL SCIENCE AND RESEARCH, Kenepuru, porirua (NZ)

(72) Inventors: Rachel Ingrid Fleming, Auckland (NZ); Meng-Han Lin, Auckland (NZ)

(21) Appl. No.: 15/563,032

(22) PCT Filed: Apr. 1, 2016
(86) PCT No.: PCT/NZ2016/050056

§ 371 Date: Sep. 29, 2017

(30) Foreign Application Priority Data
Apr. 1, 2015 (NZ) 706580

(51) Int. Cl. C12Q 1/6806; C12Q 1/6876; C12Q 1/6874

(52) U.S. Cl. CPC C12Q 1/6806; C12Q 2521/107; C12Q 1/6874; C12Q 1/6876

(57) ABSTRACT

The invention relates to a methods for detecting RNA sequences. The invention also relates to nucleotide sequences, primers, probes and microarrays.

### TECHNICAL FIELD

The technical field is applications involving detection of RNA sequences, and the use of these sequences for identification and typing of samples, in particular samples containing degraded RNA.

### BACKGROUND

The ability to accurately detect and quantify RNA abundance is a fundamental capability in molecular biology. The broad set of RNA detection methods currently available range from non-amplification methods (in situ hybridisation, microarray and NanoString nCounter), to amplification (PCR) based methods (reverse transcriptase PCR (RT-PCR) and quantitative reverse transcriptase PCR (qRT-PCR)). With the exception of RNAseq (next generation sequencing, also referred to as second generation sequencing or massively parallel sequencing), a key prerequisite of all RNA detection technology is prior knowledge of the target RNA sequence. This targeting is facilitated by oligonucleotide sequences in both non-amplification methods (probe) and amplification-based methods (primers).

Methods for PCR primer design are always evolving [1, 2] but remain based around the core criteria of specificity, thermodynamics, secondary structure, dimerisation and amplicon length [3-7]. In addition to these criteria, RT-PCR primer design (for RNA amplification) also considers exon boundary coverage to ensure amplification of only cDNA and avoid amplification of genomic DNA [8]. Amongst other experimental factors [9-14], it is widely acknowledged that PCR primer design has critical implications to target amplification, detection and quantification [3, 8, 11, 15-18].

Whilst improvements to primer design can yield performance improvements, the target molecule must also be considered. RNA is unstable and easily degraded [19-22]. Conventional methodology recommends sample RNA integrity (RIN) to be at least RIN 8 or above to ensure proper performance [23-26]. RIN values range from 10 (intact) to 1 (totally degraded). The gradual degradation of RNA is reflected by a continuous shift towards shorter RNA fragments the more degraded the RNA is. In this context shorter means that the RNA fragments are not as long as non-degraded RNA and over time the RNA fragments break down into smaller and smaller fragments.

A degree of degradation is unavoidable in situations where real-world samples must be analysed—forensic, clinical, FFPE and environmental sampling. The detrimental effects of RNA degradation on RNA detection and quantification are well documented [24, 27-30]. Currently there is no clear solution to this problem except to avoid analysing degraded RNA.

It is an object of the invention is to provide improved methods and/or materials for specific detection of RNA sequences in samples that have been subject to degradation. It is a further or alternate object of the invention to provide a method and/or materials for specific detection of RNA sequences in samples and/or at least to provide the public with a useful choice.

### SUMMARY OF THE INVENTION

The present invention provides methods for design, production and use of probes and primers that are directed to stable regions of the RNA of interest. The methods involve the use of next generation sequencing to identify stable regions of RNA of interest. Probes or primers are then designed that will hybridise to the identified stable regions.

The inventors postulated that when the next generation sequencing data shows a higher number of sequencing reads aligned to a particular region of a given RNA, then this region is more stable, or less degraded, than regions of the RNA with fewer, or no, aligned sequencing reads. RNA regions of lower sequencing read coverage were postulated to indicate regions where the transcript has degraded. The applicants have shown that targeting the stable regions they have identified for primer design, allows improved detection of the RNA relative to that shown when standard primer design approached are used.

The inventors have shown that this invention is particularly useful for detection of RNA sequence of interest in forensic samples. Detection of such RNA sequences, or RNA marker sequences, is useful in identification or typing or any given forensic sample. The invention is particularly useful for detection of such RNA marker sequences in samples that have been subjected to degradation, as is often the case for forensic samples.

The methods and materials of the invention however have wider application than just forensic samples. These materials and methods can be applied to any situation where detection of an RNA sequence in biological samples is required, and particularly in situations where the sample, or RNA within, the sample has been subjected to conditions which may result in degradation of RNA sequence of interest. For example RNA stable regions may be useful in detecting RNA and degraded RNA in a wide range of samples including the identification of human and animal pathogens, the detection of cancer, including in early diagnostics, and for the detection of invasive species for example, in biosecurity testing.

Using RNA stable regions may provide more sensitive and accurate diagnostic techniques compared to conventional methods. For example, foodborne and waterborne Hepatitis A Virion (HAV) is a leading cause of human viral infections. HAV poorly replicates in cell cultures and to detect HAV, a number of RT-PCR assays have been developed that detect small amounts of viral RNA in environmental sources, food samples and clinical specimens. The sensitivity and specificity of these RT-PCR assays are dependent on primer design and the presence of the target. Such primer designs do not consider RNA stability to determine the primer annealing sites. The small amounts of viral RNA from environmental, food or clinical specimens would be difficult to detect using conventional methods. Identifying the stable regions of viral RNA and designing primers to these targets may improve the sensitivity and specificity of these assays. (Molecular Detection of Foodborne Pathogens. Ed. Dongyou Liu (2009) 64-65, and The detection of bacteria in food, using RNA-aptamers (Maeng et al.), RT-PCR methods (Law, et al.)).

Methods

In a first aspect the invention provides a method for the detection of an RNA sequence in a sample, the method including the steps:
a) providing a sample, and
b) detecting the RNA sequence using at least one primer or probe complementary to a stable region of the RNA sequence.

Preferably the stable region of the RNA sequence has been identified using RNA sequencing of the sample.

Preferably the stable region of the RNA sequence has been identified as a region in the RNA sequence which has more aligned sequencing reads than another region, or regions, of the same RNA sequence.

Preferably the stable region is selected from the group comprising SEQ ID NO:6 to SEQ ID NO:10 and SEQ ID NO:39 to SEQ ID NO:56 or a compliment of anyone thereof.

Preferably the primer is selected from the group comprising SEQ ID NO:11 to SEQ ID NO:20 or compliment of anyone thereof.

Preferably the probe is selected from the group comprising SED ID NO:57 to SEQ ID NO:92 or compliment of anyone thereof.

Preferably the sample is a biological tissue sample.

Preferably the sample is a solid sample.

Preferably the sample is a liquid sample.

Preferably the sample is from an internal organ.

Preferably the sample is selected from the group comprising heart, brain, liver, fat, muscle, gastrointestinal tract, lung and bone.

Preferably the sample is a forensic sample.

Preferably the forensic sample is selected from the group comprising blood, buccal, saliva, menstrual blood, skin, semen and vaginal fluid.

Preferably the RNA is extracted from the sample prior to the detecting step.

Preferably the RNA sequence is detected directly.

Preferably the RNA sequence is detected indirectly.

Preferably the RNA sequence is detected indirectly by detection of a complementary DNA (cDNA) corresponding to the RNA sequence.

In another aspect the invention provides a method of typing a sample including RNA, the method including the steps:
a) providing a sample including RNA;
b) detecting one or more stable RNA sequences in the sample using at least one primer or probe complementary to the one or more stable region of the RNA;

wherein the stable RNA sequence is specific for the type of sample; and

wherein detecting the stable RNA sequence indicates the type of sample.

Preferably the stable region of the RNA sequence has been identified using RNA sequencing of the sample.

Preferably the stable region of the RNA sequence has been identified as a region in the RNA sequence which has more aligned sequencing reads than another region, or regions, of the same RNA sequence.

Preferably the stable region is selected from the group comprising SEQ ID NO:6 to SEQ ID NO:10 and SEQ ID NO:39 to SEQ ID NO:56 or a compliment of anyone thereof.

Preferably the primer is selected from the group comprising SEQ ID NO:11 to SEQ ID NO:20.

Preferably the probe is selected from the group comprising SED ID NO:57 to SEQ ID NO:92 or compliment of anyone thereof.

Preferably the sample is a biological tissue sample.

Preferably the sample is a solid sample.

Preferably the sample is a liquid sample.

Preferably the sample is from an internal organ.

Preferably the sample is selected from the group comprising heart, brain, liver, fat, muscle, gastrointestinal tract, lung and bone.

Preferably the sample is a forensic sample.

Preferably the forensic sample is selected from the group comprising blood, buccal, saliva, menstrual blood, skin, semen and vaginal fluid.

Preferably the RNA is extracted from the sample prior to the detecting step. Preferably the RNA sequence is detected directly.

Preferably the RNA sequence is detected indirectly.

Preferably the RNA sequence is detected indirectly by detection of a complementary DNA (cDNA) corresponding to the RNA sequence.

In another aspect the invention provides method of typing a sample including degraded RNA, the method including the steps:
a) providing a sample including degraded RNA;
b) detecting one or more stable RNA sequences in the sample using at least one primer or probe complementary to the one or more stable region of the degraded RNA;

wherein the stable RNA sequence is specific for the type of sample; and

wherein detecting the target RNA sequence indicates the type of sample.

Preferably the stable region of the RNA sequence has been identified using RNA sequencing of the sample.

Preferably the stable region of the RNA sequence has been identified as a region in the RNA sequence which has more aligned sequencing reads than another region, or regions, of the same RNA sequence.

Preferably the stable region is selected from the group comprising SEQ ID NO:6 to SEQ ID NO:10 and SEQ ID NO:39 to SEQ ID NO:56 or a compliment of anyone thereof.

Preferably the primer is selected from the group comprising SEQ ID NO:11 to SEQ ID NO:20.

Preferably the probe is selected from the group comprising SED ID NO:57 to SEQ ID NO:92 or compliment of anyone thereof.

Preferably the sample is a biological tissue sample.

Preferably the sample is a solid sample.

Preferably the sample is a liquid sample.

Preferably the sample is from an internal organ.

Preferably the sample is selected from the group comprising heart, brain, liver, fat, muscle, gastrointestinal tract, lung and bone.

Preferably the sample is a forensic sample.

Preferably the forensic sample is selected from the group comprising blood, buccal, saliva, menstrual blood, skin, semen and vaginal fluid.

Preferably the RNA is extracted from the sample prior to the detecting step.

Preferably the RNA sequence is detected directly.

Preferably the RNA sequence is detected indirectly.

Preferably the RNA sequence is detected indirectly by detection of a complementary DNA (cDNA) corresponding to the RNA sequence.

In another aspect the invention provides a method for the identification of a stable region in RNA in a sample, the method comprising:
a) providing a sample including RNA,
b) isolating total RNA from the sample,
c) removing DNA from the sample
d) generating cDNA complementary to the RNA in the sample,
e) sequencing the cDNA

wherein the stable region of the RNA sequence is identified as a region in the RNA sequence which has more aligned sequencing reads than another region, or regions, of the same RNA sequence.

Preferably the RNA is degraded.

Preferably the RNA has an RIN value of less than 8.

Preferably the stable region of the RNA sequence is identified as a region in the RNA sequence which has more aligned sequencing reads than another region, or regions, of the same RNA sequence.

In one embodiment of the methods, RNA is extracted from the sample prior to the detecting step.

The RNA sequence may be detected directly.

Alternatively the RNA sequence may be detected indirectly, via detection of a complementary DNA (cDNA) corresponding to the RNA sequence.

The cDNA sequence may be reverse transcribed from the RNA sequence.

Detection with Primer

In one embodiment the RNA sequence is detected using a primer.

Preferably the RNA sequence is detected using two primers.

Preferably both of the primers correspond to, are complementary to, or are capable of hybridising to, a sequence within the stable region.

In these embodiments the primers are used to amplify the part of the stable region bound by the primers.

In one embodiment amplification is by a polymerase chain reaction (PCR) method.

In one embodiment the PCR method is selected from standard PCR, reverse transcriptase (RT)-PCR, and quantitative reverse transcriptase PCR (qRT-PCR)

Detection with Probe

In a further embodiment the RNA sequence is detected using a probe.

Preferably the probe corresponds to, or is complementary to, a sequence within the stable region.

Sample

In one embodiment the sample is a biological tissue sample.

In a further embodiment the sample is a solid sample. In a further embodiment the sample is a liquid sample.

Preferred samples include RNA from internal organs. Preferred internal organs include heart, brain and liver.

Other preferred samples include RNA from fat, muscle, gastrointestinal tract, lungs, and bone samples.

In a preferred embodiment the sample is a forensic sample.

Preferred forensic samples include: blood, buccal/saliva, menstrual blood, skin, semen and vaginal fluid.

In one embodiment the sample is circulatory blood. In a further embodiment the sample is oral mucosa/saliva (buccal). In a further embodiment the sample is menstrual blood.

In a further embodiment the sample is skin. In a further embodiment the sample is semen. In a further embodiment the sample is vaginal fluid. In a further embodiment the sample is an internal organ.

In another embodiment, the sample is from an environmental or processing source.

In a preferred embodiment the sample is used for the detection of invasive species for example, in biosecurity testing.

Field samples may include plant (partial leaf, cuttings, sap/exudate or root material), animal (biological fluid/biopsy), human (biological fluid/biopsy) and marine/aquaculture material (marine animals, fish, plant, algae and water quality). The non-pristine nature and limited abundance of field samples make the detection of target RNA from invasive species (virus and other microorganisms) difficult due to limits of detection sensitivity, subsequently limiting specificity.

Markers within Sample

In one embodiment the RNA sequence is encoded by a marker gene specific for the type of sample.

That is, the expression of the RNA sequence, or presence of the RNA sequence, in the sample, is diagnostic for the type of sample.

In one embodiment, when the sample is circulatory blood, the marker gene is selected from:
Hemoglobin delta (HBD),
Solute carrier family 4 (anion exchanger), member 1 (Diego blood group) (SLC4A1),
Glycophorin A (MNS blood group) (GYPA),
Hemoglobin, beta (HBB), and
Pro-platelet basic protein (chemokine (C-X-C motif) ligand 7) (PPBP).

In a further embodiment when the sample is oral mucosa/saliva (buccal), the marker gene is selected from:
the saliva marker Histatin 3 (HTN3),
Proline-rich protein BstNI subfamily 4 (PRB4), and
Statherin (STATH)

In a further embodiment when the sample is menstrual blood, the marker genes is selected from:
Matrix metallopeptidase 11 (MMP11),
Matrix metallopeptidase 10 (strom elysin 2) (MMP10),
Matrix metallopeptidase 3 (MMP3),
Matrix metallopeptidase 7 (MMP7), and
Stanniocalcin 1 (STC1).

In a further embodiment when the sample is vaginal fluid, the marker genes is Chemokine (C-X-C motif) ligand 8 (CXCL8).

In a further embodiment the RNA sequence encoded by the marker gene corresponds to the cDNA sequence of any one of SEQ ID NO: 1 to 5 and 21 to 38.

In a further embodiment the stable region of the RNA sequence corresponds to the cDNA sequence of any one of SEQ ID NO: 6 to 10 and 39 to 56.

In a further aspect the invention provides a nucleotide sequence comprising at least 5 nucleotides with at least 70% identity to a sequence selected from SEQ ID NO:6 to SEQ ID NO:10 or a compliment thereof, or a sequence selected from SEQ ID NO:39 to SEQ ID NO:56 or a compliment thereof.

In a further aspect the invention provides a nucleotide sequence comprising at least 5 nucleotides of a sequence selected from SEQ ID NO:6 to SEQ ID NO:10 or a compliment thereof, or a sequence selected from SEQ ID NO:39 to SEQ ID NO:56 or a compliment thereof.

In a further aspect the invention provides a nucleotide sequence is selected from any one of SEQ ID NO:11 to SEQ ID NO:20.

In a further aspect the invention provides a nucleotide sequence comprising at least 10 nucleotides with at least 70% identity to a sequence selected from SEQ ID NO:6 to SEQ ID NO:10 or a compliment thereof, or a sequence selected from SEQ ID NO:39 to SEQ ID NO:56 or a compliment thereof.

In a further aspect the invention provides a nucleotide sequence comprising at least 10 nucleotides of a sequence selected from SEQ ID NO:6 to SEQ ID NO:10 or a compliment thereof, or a sequence selected from SEQ ID NO:39 to SEQ ID NO:56 or a compliment thereof.

In a further aspect the invention provides a nucleotide sequence selected from any one of SEQ ID NO:57 to SEQ ID NO:92

In a further aspect the invention provides the use of a nucleotide sequence defined above in the typing of a sample including RNA.

Primers

In a further embodiment detection involves use of a primer capable of hybridising to the stable region of the RNA sequence, or a cDNA corresponding to the stable region or a complement thereof.

In a further embodiment detection involves use of a primer comprising a sequence of at least 5 nucleotides with at least 70% identity to any part of the sequence of any one of SEQ ID NO: 6 to 10 and 39 to 56 or a complement thereof.

In a further embodiment the primer consists of a sequence of at least 5 nucleotides with at least 70% identity to the sequence of any one of SEQ ID NO: 6 to 10 and 39 to 56, or a complement thereof.

In a further embodiment the primer comprises a sequence of at least 5 nucleotides of the sequence of any one of SEQ ID NO: 6 to 10 and 39 to 56, or a complement thereof.

In a further embodiment the primer consists of a sequence of at least 5 nucleotides of the sequence of any one of SEQ ID NO: 6 to 10 and 39 to 56, or a complement thereof.

In a further embodiment the primer comprises a sequence selected from any one of SEQ ID NO: 11 to 20.

In a further embodiment the primer consists of a sequence selected from any one of SEQ ID NO: 11 to 20.

In a further embodiment the primer consists of a label or tag attached to a sequence selected from any one of SEQ ID NO: 11 to 20.

Probes

In a further embodiment detection involves use of a probe capable of hybridising to the stable region of the RNA sequence, or a cDNA corresponding to the stable region or a complement thereof.

In a further embodiment detection involves use of a probe comprising a sequence of at least 10 nucleotides with at least 70% identity to any part of the sequence of any one of SEQ ID NO: 6 to 10 and 39 to 56 or a complement thereof.

In a further embodiment the probe consists of a sequence of at least 10 nucleotides with at least 70% identity to the sequence of any one of SEQ ID NO: 6 to 10 and 39 to 56, or a complement thereof.

In a further embodiment the probe comprises a sequence of at least 10 nucleotides of the sequence of any one of SEQ ID NO: 6 to 10 and 39 to 56, or a complement thereof.

In a further embodiment the probe consists of a sequence of at least 10 nucleotides of the sequence of any one of SEQ ID NO: 6 to 10 and 39 to 56, or a complement thereof.

In a further embodiment the probe comprises a sequence selected from any one of SEQ ID NO: 57 to 92.

In a further embodiment the probe consists of a sequence selected from any one of SEQ ID NO: 57 to 92.

In a further embodiment the probe consists of a label or tag attached to a sequence selected from any one of SEQ ID NO: 57 to 92.

Typing a Sample

In a further aspect the invention provides a method of typing a sample, the method comprising the steps of detecting an RNA sequence in a sample by a method of the invention, wherein detecting the RNA sequence marker indicates the type of sample.

The method may involve using just one pair of primers, or a single probe, to type the sample. Alternatively multiple pairs of primers, or multiple probes, may be used.

Typing sample by Multiplex PCR

In one embodiment multiplex PCR is performed with multiple primers, at least one of which is diagnostic for the type of sample.

Preferably multiplex PCR is performed using at least 4, more preferably at least 5, more preferably at least 6, more preferably at least 7, more preferably at least 8, more preferably at least 9, more preferably at least 10, more preferably at least 11, more preferably at least 12, more preferably at least 13, more preferably at least 14, more preferably at least 15, more preferably at least 16, more preferably at least 17, more preferably at least 18, more preferably at least 19, more preferably at least 20, more preferably at least 21, more preferably at least 22, more preferably at least 23, more preferably at least 24, more preferably at least 25, more preferably at least 26, more preferably at least 27, more preferably at least 28, more preferably at least 29, more preferably at least 30 primers of the invention.

In a preferred embodiment, the method of the invention results in amplification of a product, or a hybridisation event, that would not occur in nature, or in the absence of the method of the invention.

Products

Primers

In a further embodiment the invention provides a primer capable of hybridising to the stable region of the RNA sequence, or a cDNA corresponding to the stable region or a complement thereof.

In a further embodiment the invention provides a primer comprising a sequence of at least 5 nucleotides with at least 70% identity to any part of the sequence of any one of SEQ ID NO: 6 to 10 and 39 to 56 or a complement thereof.

In a further embodiment the primer consists of a sequence of at least 5 nucleotides with at least 70% identity to the sequence of any one of SEQ ID NO: 6 to 10 and 39 to 56, or a complement thereof.

In a further embodiment the primer comprises a sequence of at least 5 nucleotides of the sequence of any one of SEQ ID NO: 6 to 10 and 39 to 56, or a complement thereof. In a further embodiment the primer consists of a sequence of at least 5 nucleotides of the sequence of any one of SEQ ID NO: 6 to 10 and 39 to 56, or a complement thereof. In a further embodiment the primer comprises a sequence selected from any one of SEQ ID NO: 11 to 20, or a complement thereof.

In a further embodiment the primer consists of a sequence selected from any one of SEQ ID NO: 11 to 20, or a complement thereof.

In a further embodiment the primer consists of a label or tag attached to a sequence selected from any one of SEQ ID NO: 11 to 20, or a complement thereof.

In a further embodiment the labelled or tagged primer is not found in nature.

The primers of the invention can be used on microarrays or chips or like products for the detection of RNA sequences.

Kit of Primers

In a further embodiment the invention provides a kit comprising at least one primer of the invention.

Preferably the kit comprises at least 2, more preferably at least 3, more preferably at least 4, more preferably at least 5, more preferably at least 6, more preferably at least 7, more preferably at least 8, more preferably at least 9, more preferably at least 10, more preferably at least 11, more preferably at least 12, more preferably at least 13, more preferably at least 14, more preferably at least 15, more preferably at least 16, more preferably at least 17, more preferably at least 18, more preferably at least 19, more preferably at least 20, more preferably at least 21, more preferably at least 22, more preferably at least 23, more preferably at least 24, more preferably at least 25, more preferably at least 26, more preferably at least 27, more preferably at least 28, more preferably at least 29, more preferably at least 30 primers of the invention. In one embodiment the kit also comprises instructions for use.

Probes

In a further embodiment the invention provides a probe capable of hybridising to the stable region of the RNA sequence, or a cDNA corresponding to the stable region or a complement thereof.

In a further embodiment the invention provides a probe comprising a sequence of at least 10 nucleotides with at least 70% identity to any part of the sequence of any one of SEQ ID NO: 6 to 10 and 39 to 56 or a complement thereof.

In a further embodiment the probe consists of a sequence of at least 10 nucleotides with at least 70% identity to the sequence of any one of SEQ ID NO: 6 to 10 and 39 to 56, or a complement thereof.

In a further embodiment the probe comprises a sequence of at least 10 nucleotides of the sequence of any one of SEQ ID NO: 6 to 10 and 39 to 56, or a complement thereof.

In a further embodiment the probe consists of a sequence of at least 10 nucleotides of the sequence of any one of SEQ ID NO: 6 to 10 and 39 to 56, or a complement thereof.

In a further embodiment the probe comprises a sequence selected from any one of SEQ ID NO: 57 to 92, or a complement thereof.

In a further embodiment the probe consists of a sequence selected from any one of SEQ ID NO: 57 to 92, or a complement thereof.

In a further embodiment the probe consists of a label or tag attached to a sequence selected from any one of SEQ ID NO: 57 to 92, or a complement thereof.

In a further embodiment the labelled or tagged probe is not found in nature.

The primers of the invention can be used on microarrays or chips or like products for the detection of RNA sequences.

Kit of Probes

In a further embodiment the invention provides a kit comprising at least one probe of the invention.

Preferably the kit comprises at least 2, more preferably at least 3, more preferably at least 4, more preferably at least 5, more preferably at least 6, more preferably at least 7, more preferably at least 8, more preferably at least 9, more preferably at least 10, more preferably at least 11, more preferably at least 12, more preferably at least 13, more preferably at least 14, more preferably at least 15, more preferably at least 16, more preferably at least 17, more preferably at least 18, more preferably at least 19, more preferably at least 20, more preferably at least 21, more preferably at least 22, more preferably at least 23, more preferably at least 24, more preferably at least 25, more preferably at least 26, more preferably at least 27, more preferably at least 28, more preferably at least 29, more preferably at least 30 probes of the invention.

In one embodiment the kit also comprises instructions for use.

MicroArrays

In another aspect the invention provides a microarray comprising a sequence of at least 5 nucleotides with at least 70% identity to any part of the sequence of any one of SEQ ID NO:6 to SEQ ID NO:10 or a complement thereof.

In another aspect the invention provides a microarray comprising a sequence of at least 5 nucleotides of a sequence of any one of SEQ ID NO:6 to SEQ ID NO:10 or a complement thereof.

In another aspect the invention provides a microarray comprising a sequence of at least 10 nucleotides of a sequence with at least 70% identify to any part of the sequence of any one of SEQ ID NO:6 to SEQ ID NO:10 or a complement thereof.

In another aspect the invention provides a microarray comprising a sequence of at least 10 nucleotides of a sequence of any one of SEQ ID NO: 6 to SEQ ID NO: 10 or a complement thereof.

In another aspect the invention provides a microarray comprising a sequence of at least 5 nucleotides with at least 70% identity to any part of the sequence of any one of SEQ ID NO: 39 to SEQ ID NO:56 or a complement thereof.

In another aspect the invention provides a microarray comprising a sequence of at least 5 nucleotides of a sequence of any one of SEQ ID NO: 39 to SEQ ID NO:56 or a complement thereof.

In another aspect the invention provides a microarray comprising a sequence of at least 10 nucleotides with at least 70% identity to any part of the sequence of any one of SEQ ID NO: 39 to SEQ ID NO:56 or a complement thereof.

In another aspect the invention provides a microarray comprising a sequence of at least 5 nucleotides with at least 70% identity to any part of the sequence of any one of SEQ ID NO:11 to SEQ ID NO:20 or a complement thereof.

In another aspect the invention provides a microarray comprising a sequence of at least 5 nucleotides of a sequence of any one of SEQ ID NO:11 to SEQ ID NO:20 or a complement thereof.

In another aspect the invention provides a microarray comprising a sequence of at least 10 nucleotides of a sequence with at least 70% identify to any part of the sequence of any one of SEQ ID NO:11 to SEQ ID NO:20 or a complement thereof.

In another aspect the invention provides a microarray comprising a sequence of at least 10 nucleotides of a sequence of any one of SEQ ID NO: 11 to SEQ ID NO: 20 or a complement thereof.

In another aspect the invention provides a microarray comprising a sequence of at least 5 nucleotides with at least 70% identity to any part of the sequence of any one of SEQ ID NO:57 to SEQ ID NO:92 or a complement thereof.

In another aspect the invention provides a microarray comprising a sequence of at least 5 nucleotides of a sequence of any one of SEQ ID NO:57 to SEQ ID NO:92 or a complement thereof.

In another aspect the invention provides a microarray comprising a sequence of at least 10 nucleotides of a sequence with at least 70% identify to any part of the sequence of any one of SEQ ID NO:57 to SEQ ID NO:92 or a complement thereof.

In another aspect the invention provides a microarray comprising a sequence of at least 10 nucleotides of a sequence of any one of SEQ ID NO:57 to SEQ ID NO:92 or a complement thereof.

Preferably the sequence comprises at least 5, more preferably at least 10, more preferably at least 15, more preferably at least 20, more preferably at least 25, more preferably at least 30, more preferably at least 35, more preferably at least 40, more preferably at least 45, more preferably at least 50, more preferably at least 55, more preferably at least 60, more preferably at least 65, more preferably at least 70, more preferably at least 75, more preferably at least 80, more preferably at least 85, more preferably at least 90, more preferably at least 95, more preferably at least 100, more preferably at least 120, more preferably at least 140, more preferably at least 160, more preferably at least 180, more preferably at least 200, more preferably at least 240, more preferably at least 250 nucleotides of the sequences of the invention.

Tables 1 and 2 below show exemplary marker genes, cDNA sequences corresponding to the mRNA encoded by the marker genes, cDNA sequences corresponding to the stable regions of the RNA sequences, and primers and probes that hybridise to the stable regions that are useful for detecting the marker genes, particularly in degraded samples.

Those skilled in the art would understand how to select the appropriate probes or primers for detecting any of the listed markers, based on the information in Tables 1 and 2, and elsewhere in the specification.

It will be understood to those skilled in the art that once a stable region has been identified, a probe or primer can be produced that can hybridise to any part of that stable region. The probes and primers mentioned herein are given as examples only to demonstrate that the stable regions can be used to identify and type degraded RNA. Any primer or probe that is complementary to the stable region would be suitable in the methods of the invention.

TABLE 1
Sequences of marker genes, cDNA corresponding to RNA encoded by marker
gene, cDNA corresponding to stable region of RNA and primers.

| | | | cDNA | | | |
|---|---|---|---|---|---|---|
| | | | encoded | Stable | Forward | Reverse |
| | | | by RNA | region | Primer | Primer |
| | | Marker Gene | (SEQ | (SEQ | (SEQ | (SEQ |
| Sample | Marker Gene | Accession No. | ID NO) | ID NO) | ID NO) | ID NO) |
| Circulatory | Hemoglobin delta | NM_000519 | 1 | 6 | 11 | 12 |
| blood | (HBD) | | | | | |
| Circulatory | Solute carrier family | NM_000342.3 | 2 | 7 | 13 | 14 |
| blood | 4 (anion exchanger), | | | | | |
| | member 1 (Diego blood | | | | | |
| | group) (SLC4A1). | | | | | |
| Oral mucosa/ | Histatin 3 (HTN3) | NM_000200.2 | 3 | 8 | 15 | 16 |
| saliva (buccal) | | | | | | |
| Menstrual | Matrix metallopeptidase | NM_005940.3 | 4 | 9 | 17 | 18 |
| blood | 11 (MMP11) | | | | | |
| Reference | Ubiquitin-conjugating | NM_003339.2 | 5 | 10 | 19 | 20 |
| gene | enzyme E2D 2 (UBE2D2) | | | | | |

TABLE 2
Sequences of marker genes, cDNA corresponding to RNA encoded by marker
gene, cDNA corresponding to stable region of RNA and probes.

| | | | | Stable | Capture | Target |
|---|---|---|---|---|---|---|
| | | | RNA | region | Probe | Probe |
| | | | SEQ | SEQ | SEQ | SEQ |
| Sample | Marker Gene | Accession No. | ID NO: | ID NO: | ID NO: | ID NO: |
| Reference | Actin, beta (ACTB) | NM_001101.2 | 21 | 39 | 57 | 58 |
| gene | | | | | | |
| Vaginal fluid | Chemokine (C-X-C | NM_000584.3 | 22 | 40 | 59 | 60 |
| | motif) ligand 8 (CXCL8) | | | | | |
| Oral mucosa/ | Follicular dendritic cell | NM_152997.3 | 23 | 41 | 61 | 62 |
| saliva (buccal) | secreted protein (FDCSP) | | | | | |
| Reference | Glucose-6-phosphate | NM_000402.4 | 24 | 42 | 63 | 64 |
| gene | dehydrogenase (G6PD) | | | | | |
| Reference | Glyceraldehyde-3- | NM_002046.3 | 25 | 43 | 65 | 66 |
| gene | phosphate | | | | | |
| | dehydrogenase (GAPDH) | | | | | |
| Ciculatory | Glycophorin A (MNS | NM_002099.6 | 26 | 44 | 67 | 68 |
| blood | blood group) (GYPA) | | | | | |
| Ciculatory | Hemoglobin, beta | NM_000518.4 | 27 | 45 | 69 | 70 |
| blood | (HBB) | | | | | |
| Menstrual | Matrix metallopeptidase | NM_002425.1 | 28 | 46 | 71 | 72 |
| blood | 10 (stromelysin 2) (MMP10) | | | | | |
| Menstrual | Matrix metallopeptidase | NM_005940.3 | 29 | 47 | 73 | 74 |
| blood | 11 (MMP11) | | | | | |
| Menstrual | Matrix metallopeptidase | NM_002422.3 | 30 | 48 | 75 | 76 |
| blood | 3 (MMP3) | | | | | |
| Menstrual | Matrix metallopeptidase | NM_002423.3 | 31 | 49 | 77 | 78 |
| blood | 7 (MMP7) | | | | | |
| Ciculatory | Pro-platelet basic protein | NM_002704.3 | 32 | 50 | 79 | 80 |
| blood | (chemokine (C-X-C motif) | | | | | |
| | ligand 7) (PPBP) | | | | | |
| Oral mucosa/ | Proline-rich protein | NM_001261399.1 | 33 | 51 | 81 | 82 |
| saliva (buccal) | BstNI subfamily 4 (PRB4) | | | | | |
| Ciculatory | Solute carrier family 4 | NM_000342.3 | 34 | 52 | 83 | 84 |
| blood | (anion exchanger), | | | | | |
| | member 1 (Diego blood | | | | | |
| | group) (SLC4A1) | | | | | |
| Oral mucosa/ | Statherin (STATH) | NM_001009181.1 | 35 | 53 | 85 | 86 |
| saliva (buccal) | | | | | | |
| Menstrual | Stanniocalcin 1 | NM_003155.2 | 36 | 54 | 87 | 88 |
| blood | (STC1) | | | | | |
| Reference | Transcription | NM_006756.3 | 37 | 55 | 89 | 90 |
| gene | elongation factor A | | | | | |
| | (SII), 1 (TCEA1) | | | | | |
| Reference | Ubiquitin-conjugating | NM_181838.1 | 38 | 56 | 91 | 92 |
| gene | enzyme E2D 2 (UBE2D2) | | | | | |

Those skilled in the art will understand the relationship between marker genes, the mRNA encoded by the marker genes, and the stable regions within the mRNA. Those skilled in the art will understand that the sequences presented are DNA sequences corresponding to the mRNA or stable regions within the mRNA.

### DETAILED DESCRIPTION OF THE INVENTION

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

The term “comprising” as used in this specification and claims means “consisting at least in part of”; that is to say when interpreting statements in this specification and claims which include “comprising”, the features prefaced by this term in each statement all need to be present but other features can also be present. Related terms such as “comprise” and “comprised” are to be interpreted in similar manner. However, in preferred embodiments comprising can be replaced with consisting.

As used here, the term “RNA” means messenger RNA, small RNA, microRNA, non-coding

RNA, long non-coding RNA, ribosomal RNA, small nucleolar RNA, transfer RNA and all other RNA species and sequences.

As used herein, the term “stable region” means a region or regions in an RNA sequence which has more aligned sequencing reads than another region, or regions, of the same RNA sequence.

As used herein the term “degraded RNA” refers to is RNA that is no longer intact. In other words, the theoretical full length RNA, as annotated or predicted in sequence databases, is no longer intact. The full length RNA may be fragmented and/or some nucleotides are no longer present. This may occur at any position along the RNA sequence.

One measure of the level of degradation in an RNA sequence is the RNA integrity (RIN) value. RIN values range from 10 (fully intact) to 1 (totally degraded). Conventional methodology recommends sample RNA integrity (RIN) to be at least RIN 8 or above to ensure proper performance of RNA analysis as previously discussed.

The inventors have surprisingly found that stable regions in RNA specific to sample types can survive degradation and be present in samples that have RIN values of less than 8, including samples that have RIN values of 0 (i.e. the sample is so degraded that a RIN value is unable to be determined). These stable regions can be used to type samples using primers and probes. The stable regions can be used to type samples having RIN values of less than 8 but also, as those stable regions will also be present in other equivalent samples having RIN values of greater than 8, the stable regions can be used to type samples if they have RIN values of greater than 8 as well.

The present invention provides improved materials and methods for detecting RNA sequences in samples. The method involves using RNA sequencing to identify stable regions of RNA of interest on the basis of RNA sequencing data showing multiple aligned reads over the regions.

The method of the invention then involves producing probes or primers targeting the stable regions. The method allows for improved detection of such RNA sequences, particularly in samples in which the RNA is, or has been, subjected to degradation.

RNA Degradation

Whilst improvements to primer or probe design design can yield performance improvements in amplification and hybridisation methods, the target molecule must also be considered. RNA is unstable and easily degraded [19-22]. Conventional methodology recommends sample RNA integrity (RIN) to be at least RIN 8 or above to ensure proper performance [23-26].

A degree of degradation is unavoidable in situations where real-world samples must be analysed—forensic, clinical, FFPE and environmental sampling. The detrimental effects of RNA degradation on RNA detection and quantification are well documented [24, 27-30].

The methods and materials of the invention allow for improved detection of RNA sequences of interest, particularly when RNA samples have been degraded. This allows typing of samples that contain that degraded RNA, including samples having a RIN value less than 8. This is particularly surprising as prior to the present invention it was generally considered that detection and typing of degraded RNA sequences where RIN was less than 8, was not able to be achieved to an acceptable performance value.

RIN values range from 10 (intact) to 1 (totally degraded). The gradual degradation of RNA is reflected by a continuous shift towards shorter RNA fragments the more degraded the RNA is. Where the RIN value is less than 1, this signifies that RNA is degraded beyond detection.

While the inventors have found that while the probes and primers of the invention are useful in detecting and typing the source of degraded RNA including RNA having a RIN value less than 8, the probes and primers of the invention can also be used to detect and type the source of RNA having a RIN value of 8-10. That is, the primers and probes of the invention also allow the detection and typing of RNA irrespective of the RIN value.

In one embodiment the methods of the invention works, or allow for RNA marker detection, when RNA integrity (RIN) is less than RIN 8, more preferably less than RIN 7, more preferably less than RIN 6, more preferably less than RIN 5, more preferably less than RIN 4, more preferably less than RIN 3, more preferably less than RIN 2, more preferably less that than 1. The inventors have also found that the methods of the invention can be used to type RNA where RIN is undetermined (beyond detection).

Applications for the Methods and Materials of the Invention

The methods and materials of the invention may be applied to any process involving detection of RNA, particularly in situations where degradation of target RNA is a problem.

The broad set of RNA detection methods currently available range from non-amplification methods (in situ hybridisation, microarray and NanoString nCounter), to amplification (PCR) based methods (reverse transcriptase PCR (RT-PCR) and quantitative reverse transcriptase PCR (qRT-PCR), and RNA-aptamers.

In Situ Hybridisation

In situ hybridization (ISH) is a type of hybridization that uses a labelled complementary DNA or RNA strand (i.e., probe) to localize a specific DNA or RNA sequence in a portion or section of tissue (in situ), or, if the tissue is small enough (e.g., plant seeds, Drosophila embryos), in the entire tissue (whole mount ISH), in cells, and in circulating tumor cells (CTCs). This is distinct from immunohistochemistry, which usually localizes proteins in tissue sections.

In situ hybridization is a powerful technique for identifying specific mRNA species within individual cells in tissue sections, providing insights into physiological processes and disease pathogenesis. However, in situ hybridization requires that many steps be taken with precise optimization for each tissue examined and for each probe used. In order to preserve the target mRNA within tissues, it is often required that crosslinking fixatives (such as formaldehyde) be used.

Degradation of target RNA is a problem in ISH experiments. The methods of the invention provide a solution to this problem by targeting stable regions within target RNA of interest.

Microarray

A DNA microarray (also commonly known as DNA chip or biochip) is a collection of microscopic DNA spots attached to a solid surface. Scientists use DNA microarrays to measure the expression levels of large numbers of genes simultaneously or to genotype multiple regions of a genome. Each DNA spot contains picomoles (10-12 moles) of a specific DNA sequence, known as probes (or reporters or oligos). These can be a short section of a gene or other DNA element that are used to hybridize a cDNA or cRNA (also called anti-sense RNA) sample (called target) under high-stringency conditions. Probe-target hybridization is usually detected and quantified by detection of fluorophore-, silver-, or chemiluminescence-labeled targets to determine relative abundance of nucleic acid sequences in the target.

The present invention has application for microarray analysis of tissues that are subject to degradation. By designing probes, to include on the microarray chip, that target stable regions of RNA (according to the present invention), the microarray analysis may provide a more realistic representation of the in vivo expression profile, that is not so skewed by degradation after RNA is extracted from the tissue sample. Such chips would also be able to be used to screen samples containing RNA, including degraded RNA, in order to type the source of that RNA as has been previously described.

NanoString nCounter

NanoString's nCounter technology is a variation on the DNA microarray and was invented and patented by Krassen Dimitrov and Dwayne Dunaway. It uses molecular “barcodes” and microscopic imaging to detect and count up to several hundred unique RNAs in one hybridization reaction. Each color-coded barcode is attached to a single target-specific probe corresponding to a gene of interest.

The NanoString protocol includes the following steps:
Hybridization: NanoString's Technology employs two ˜50 base probes per mRNA that hybridize in solution. The reporter probe carries the signal, while the capture probe allows the complex to be immobilized for data collection.
Purification and Immobilization: After hybridization, the excess probes are removed and the probe/target complexes are aligned and immobilized in the nCounter Cartridge.
Data Collection: Sample Cartridges are placed in the Digital Analyzer instrument for data collection. Color codes on the surface of the cartridge are counted and tabulated for each target molecule.

The nCounter Analysis System: The system consists of two instruments: the Prep Station, which is an automated fluidic instrument that immobilizes CodeSet complexes for data collection, and the Digital Analyzer, which derives data by counting fluorescent barcodes. As the NanoString nCounter system is dependent on probe-target hybridisation for RNA detection and analysis, this invention has immediate application to NanoString nCounter. NanoString nCounter probe design (target hybridisation sites) are designed to conform to certain thermodynamic requirements and gives no consideration to target RNA degradation or stability. Therefore we believe that with this invention NanoString nCounter RNA detection can be vastly improved by designing probes to hybridise to stable regions in the RNA sequence.

Samples

The sample may be any type of biological sample that includes RNA.

Samples suitable for in situ hybridisation include biological tissue sections.

Preferred sample include forensic samples. Preferred forensic samples include: blood, buccal/saliva, menstrual blood, semen, skin and vaginal fluid.

Other samples include samples for cancer detection and samples for bacteria and virus detection.

The analysis of RNA abundance is used for cancer detection and typing. These analyses are based on the detection of gene expression profiles (determined from RNA analysis) of known cancer genes.

Clinical samples used for cancer detection can be degraded (formalin-fixed paraffin-embedded FFPE tissue sections or biopsy) and of limited abundance. While the methods of the invention may be used to detect any form of cancer, examples where the methods of the invention may be used are:
Gene expression analysis (RNA analysis) using biopsies taken for skin/breast tissue is used to diagnose skin/breast cancer
A pap smear (non-pristine, biological fluid) is analysed for the detection of Human papilloma virus (HPV) is used for to diagnose cervical cancer
Gene expression analysis (RNA analysis) using urine samples is used to diagnose prostate cancer

These examples all require the accurate detection of target RNA sequences from degraded and low abundance samples. These assays represent situations where the methods of the invention may increase assay sensitivity and specificity. Plant biosecurity may require the detection of invasive species of plant pathogens.

Examples include leaf material or sap/exudate sampled to detect protein-encoding genes specific for the kiwifruit vine bacterium Pseudomonas syringae pv. actinidiae (Psa); or for the detection of RNA sequences of other viral plant pathogens.

Aquaculture biosecurity may require the detection of RNA sequences indicative of invasive species such as the dinoflagellates Alexandrium cantenella and Karenia brevis; the diatom Pseudo nitzschia sp; the sea squirts Didemnum vexillum and Ciona savignyi; and the Mediterranean fan-worm Sabella spalanzanii.

These examples are situations where the use of the methods of the invention would increase assay sensitivity and specificity.

RNA Extraction

RNA extraction procedures are well known to those skilled in the art. Examples include: Acid guanidium thiocyanate-phenol-chloroform RNA extraction (Chomczynski, Piotr, and Nicoletta Sacchi. The single-step method of RNA isolation by acid guanidinium thiocyanate-phenol-chloroform extraction: twenty-something years on. Nature protocols 1(2) (2006): 581-585); magnetic bead-based RNA extraction (Berensmeier, Sonja. “Magnetic particles for the separation and purification of nucleic acids.” Applied microbiology and biotechnology 73(3) (2006): 495-504); column-based RNA purification (Matson, R. S. (2008). Microarray Methods and Protocols. Boca Raton, Fla.: CRC. pp. 7-29. ISBN 1420046659; Kumar, A. (2006). Genetic Engineering. New York: Nova Science Publishers. pp. 101-102. ISBN 159454753X); and TRIzol (TRI reagent) RNA extraction (Rio, D. C., Ares, M., Hannon, G. J., & Nilsen, T. W. Purification of RNA using TRIzol (TRI reagent). Cold Spring Harbor Protocols, (2010), pdb-prot5439).

RNA Sequencing and Stable Region Identification

RNA sequencing refers to sequencing of all RNA in a sample using what is commonly known as Next Generation Sequencing (NGS) (second generation sequencing or massively parallel sequencing; Mardis, E. R. (2008). The impact of next-generation sequencing technology on genetics. Trends in genetics, 24(3), 133-141; Metzker, M. L. (2010). Sequencing technologies—the next generation. Nature Reviews Genetics, 11 (1), 31-46; Reis-Filho, J. S. (2009). Next-generation sequencing. Breast Cancer Res, 11 (Suppl 3), S12 and Schuster, S. C. (2008). Next-generation sequencing transforms today's biology. Nature methods, 5(1), 16-18). Although different sequencing instrumentation manufacturers employ slightly different sequencing chemistry, RNA sequencing can be achieved using any of these NGS (massively parallel sequencing) technologies (Mardis, 2008 and Mutz, K. O., Heilkenbrinker, A., Lonne, M., Walter, J. G., & Stahl, F. (2013). Transcriptome analysis using next-generation sequencing. Current opinion in biotechnology, 24(1), 22-30). As there are many NGS technologies available, there are small differences in the methodology for RNA sequencing. The following is a description of how RNA sequencing using NGS works in general (Metzker, 2010):
Total RNA is extracted from the sample of interest, using a common RNA extraction method. Post-extraction processes can be used to enrich the RNA sample.
Complimentary DNA (cDNA) is then synthesised using extracted RNA. cDNA is then used as the template for RNA sequencing.
NGS uses variations of sequencing by synthesis (SBS) chemistry (Fuller, C. W., Middendorf, L. R., Benner, S. A., Church, G. M., Harris, T., Huang, X., . . . & Vezenov, D. V. (2009). The challenges of sequencing by synthesis. Nature biotechnology, 27(11), 1013-1023). With cDNA as a template, new nucleotide fragments, known as reads, are synthesised base by base, with each incorporated base recorded during sequencing (Fuller, 2009).
The data output from RNA sequencing is a list of all the reads generated, and their sequence (Fuller, 2009 and Metzker, 2010). This data undergoes quality assessment (Patel, R. K., & Jain, M. (2012). NGS QC Toolkit: a toolkit for quality control of next generation sequencing data. PloS one, 7(2), e30619). For RNA sequencing, sequencing reads are then aligned to the reference genome using a splice-aware sequence alignment algorithm (Trapnell, C., Pachter, L., & Salzberg, S. L. (2009). TopHat: discovering splice junctions with RNA-Seq. Bioinformatics, 25(9), 1105-1111).
Alignments can then be visualised using any genome browser or sequence viewing software. RNA stable regions are identified by viewing sequencing read alignments along the RNA of interest. Regions along the RNA sequence where there more reads aligned (high read coverage) are deemed to be stable regions.

Stable Regions

A stable region of an RNA sequence according to the invention is a region within any given RNA sequence that RNA sequencing data shows produces more aligned sequencing reads than at least one other region with the same RNA sequence.

In a preferred embodiment the stable region has at least 1.1× more preferably 1.2×, more preferably 1.3×, more preferably 1.4×, more preferably 1.5×, more preferably 1.6×, more preferably 1.7×, more preferably 1.8×, more preferably 1.9×, more preferably 2.0×, more preferably 2.2×, more preferably 2.4×, more preferably 2.6×, more preferably 2.8×, more preferably 3.0×, more preferably, 3.2×, more preferably 3.4×, more preferably 3.6×, more preferably 3.8×, more preferably 4.0×, more preferably 4.2×, more preferably 4.4×, more preferably 4.6×, more preferably 4.8×, more preferably 5.0× as many aligned reads than at least one other region within the same RNA sequence.

PCR-Based Methods

PCR-based methods are particularly preferred for detection of RNA sequence in the method of the invention.

General PCR approaches are well known to those skilled in the art (Mullis et al., 1994). Various other developments of the basic PCR approach may also be advantageous applied to the method of the invention. Examples are discussed briefly below.

Multiplex-PCR

Multiplex-PCR utilises multiple primer sets within a single PCR reaction to produce amplified products (amplicons) of varying sizes that are specific to different target RNA, cDNA or DNA sequences. By targeting multiple sequences at once, diagnostic information may be gained from a single reaction that otherwise would require several times the reagents and more time to perform. Annealing temperatures and primer sets are generally optimized to work within a single reaction, and produce different amplicon sizes. That is, the amplicons should form distinct bands when visualized by gel electrophoresis. Multiplex PCR can be used in the method of the invention to distinguish the type of sample it applied to in a single sample or reaction.

MLPA

Multiplex ligation-dependent probe amplification (MLPA) (U.S. Pat. No. 6,955,901) is a variation of the multiplex polymerase chain reaction that permits multiple targets to be amplified with only a single primer pair. Each probe consists of two oligonucleotides which recognise adjacent target sites on the DNA. One probe oligonucleotide contains the sequence recognised by the forward primer, the other the sequence recognised by the reverse primer. Only when both probe oligonucleotides are hybridised to their respective targets, can they be ligated into a complete probe. The advantage of splitting the probe into two parts is that only the ligated oligonucleotides, but not the unbound probe oligonucleotides, are amplified. If the probes were not split in this way, the primer sequences at either end would cause the probes to be amplified regardless of their hybridization to the template DNA. Each complete probe has a unique length, so that its resulting amplicons can be separated and identified (for example by capillary electrophoresis among other methods). Since the forward primer used for probe amplification is fluorescently labeled, each amplicon generates a fluorescent peak which can be detected by a capillary sequencer. Comparing the peak pattern obtained on a given sample with that obtained on various reference samples measures presence or absence (or the relative quantity) of each amplicon can be determined. This then indicates presence or absence (or the relative quantity) of the target sequence is present in the sample DNA. The products can also be detected using gel electrophoresis or microfluid systems such as Shimadzu MultiNA. The use of reference samples to establish presence or absence is the same. More information about MLPA is available on the World Wide Web at http://www.mlpa.com. MLPA probes may be synthesized as oligonucleotides, by methods known to those skilled in the art. MLPA probes and reagents may be commercially produced by and purchased from HRC-Holland (http://www.mlpa.com).

Quantitative PCR

Quantitative PCR (Q-PCR) is used to measure the quantity of a PCR product (commonly in real-time). Q-PCR quantitatively measures starting amounts of DNA, cDNA, or RNA. Q-PCR is commonly used to determine whether a DNA sequence is present in a sample and the number of its copies in the sample. Quantitative real-time PCR has a very high degree of precision. Q-PCR methods use fluorescent dyes, such as SYBR Green, EvaGreen or fluorophore-containing DNA probes, such as TaqMan, to measure the amount of amplified product in real time. Q-PCR is sometimes abbreviated to RT-PCR (Real Time PCR) or RQ-PCR. QRT-PCR or RTQ-PCR.

Primers

The term “primer” refers to a short polynucleotide, usually having a free 3′OH group, that is hybridized to a template and used for priming polymerization of a polynucleotide complementary to the template. Such a primer is preferably at least 5, more preferably at least 6, more preferably at least 7, more preferably at least 9, more preferably at least 10, more preferably at least 11, more preferably at least 12, more preferably at least 13, more preferably at least 14, more preferably at least 15, more preferably at least 16, more preferably at least 17, more preferably at least 18, more preferably at least 19, more preferably at least 20 nucleotides in length.

In conventional primer design for amplifying RNA marker sequences, primers are typically designed to cover exon boundaries, to prevent amplification of genomic DNA.

The invention relates to targeting stable regions of RNA transcripts, which is particularly useful when amplifying markers from degraded samples. As will be readily apparent, once a stable region is identified, that region can be used to type samples containing RNA having RIN values from 8 to 10 as well as below 8. Both options thus form part of the present invention.

In one embodiment the primer of the invention for use a method of the invention, does not span an exon boundary.

Although not preferred, in one embodiment the primer of the invention for use a method of the invention, may span an exon boundary.

Labelling of Primers

Methods for labelling primers are well known to those skilled in the art, and include: Primers can be labelled enzymatically (Davies, M. J., Shah, A., & Bruce, I. J. (2000). Synthesis of fluorescently labelled oligonucleotides and nucleic acids. Chemical Society Reviews, 29(2), 97-107.) or chemically (including automated solid-phase chemical synthesis) (Proudnikov, D., & Mirzabekov, A. (1996). Chemical methods of DNA and RNA fluorescent labeling. Nucleic acids research, 24(22), 4535-4542.).

Primers can be labelled with; a fluorescence label (fluorophore, Kutyavin, I. V., Afonina, I. A., Mills, A., Gorn, V. V., Lukhtanov, E. A., Belousov, E. S., . . . & Hedgpeth, J. (2000).

3′-minor groove binder-DNA probes increase sequence specificity at PCR extension temperatures. Nucleic Acids Research, 28(2), 655-661.)), biotin (Pon, R. T. (1991). A long chain biotin phosphoramidite reagent for the automated synthesis of 5′-biotinylated oligonucleotides. Tetrahedron letters, 32(14), 1715-1718.), or radioactive and non-radioactive labels (for example digoxigenin) (Agrawal, S., Christodoulou, C., & Gait, M. J. (1986). Efficient methods for attaching non-radioactive labels to the 5′ ends of synthetic oligodeoxyribonucleotides. Nucleic acids research, 14(15), 6227-6245.).

Primers labelled by such methods form part of the invention.

Probe-Based Methods

Probe-based methods may be applied to detect the RNA sequences in the method of the invention. Methods for hybridizing probes to target nucleic acid sequences are well known to those skilled in the art (Sambrook et al., Eds, 1987, Molecular Cloning, A Laboratory Manual, 2nd Ed. Cold Spring Harbor Press).

Probe-based methods include in situ hybridization.

The term “probe” refers to a short polynucleotide that is used to detect a polynucleotide sequence that is at least partially complementary to the probe, in a hybridization-based assay. The probe may consist of a “fragment” of a polynucleotide as defined herein. Preferably such a probe is at least 10, more preferably at least 20, more preferably at least 30, more preferably at least 40, more preferably at least 50, more preferably at least 100, more preferably at least 200, more preferably at least 300, more preferably at least 400 and most preferably at least 500 nucleotides in length.

Labelling of Probes

Methods for labelling probes are well known to those skilled in the art, and include: Probes can be labelled enzymatically (Sambrook, et al. 1987; Davies, et al., 2000) or chemically (including automated solid-phase chemical synthesis) (Proudnikov, et al. 1996).

Probes can be:
Molecular Beacon (Tyagi, S., & Kramer, F. R. (1996). Molecular beacons: probes that fluoresce upon hybridization. Nature biotechnology, (14), 303-8.),
TaqMan (Kutyavin I V, Afonina I A, Mills A, Gorn V V, Lukhtanov E A, Belousov E S, Singer M J, Walburger D K, Lokhov S G, Gall A A, Dempcy R, Reed M W, Meyer R B, Hedgpeth J (2000). 3′-minor groove binder-DNA probes increase sequence specificity at PCR extension temperatures. Nucleic Acids Research, 28(2), 655-661.
Scorpion (R Carters, R., Ferguson, J., Gaut, R., Ravetto, P., Thelwell, N., & Whitcombe, D. (2008). Design and use of scorpions fluorescent signaling molecules. In Molecular beacons: Signalling nucleic acid probes, methods, and protocols (pp. 99-115). Humana Press.
In situ hybridization probes—Eisel, D.; Grünewald-Janho, S.; Krushen, B., ed. (2002). DIG Application Manual for Nonradioactive in situ Hybridization (3rd ed.). Penzberg: Roche Diagnostics.
Radioactive and non-radioactive (Simmons, D. M., Arriza, J. L., & Swanson, L. W. (1989). A complete protocol for in situ hybridization of messenger RNAs in brain and other tissues with radio-labeled single-stranded RNA probes. Journal of Histotechnology, 12(3), 169-181; Agrawal, S., Christodoulou, C., & Gait, M. J. (1986). Efficient methods for attaching non-radioactive labels to the 5′ ends of synthetic oligodeoxyribonucleotides. Nucleic acids research, 14(15), 6227-6245.).

Probes labelled by such methods form part of the invention.

Polynucleotides

The term “polynucleotide(s),” as used herein, means a single or double-stranded deoxyribonucleotide or ribonucleotide polymer of any length but preferably at least 5 nucleotides, and include as non-limiting examples, coding and non-coding sequences of a gene, sense and antisense sequences complements, exons, introns, genomic DNA, cDNA, pre-mRNA, mRNA, rRNA, siRNA, miRNA, tRNA, naturally occurring DNA or RNA sequences, synthetic RNA and DNA sequences, and fragments thereof. In one embodiment the nucleic acid is isolated, that is separated from its normal cellular environment. The term “nucleic acid” can be used interchangeably with “polynucleotide”.

Methods for Extracting Nucleic Acids

Methods for extracting nucleic acids are well-known to those skilled in the art (Sambrook et al., Eds, 1987, Molecular Cloning, A Laboratory Manual, 2nd Ed. Cold Spring Harbor Press).

Specialised extraction procedures can optionally be applied depending on the sample type, as discussed in the example section. For example, RNA from forensic type samples can be extracted using a DNA-RNA co-extraction method, as described by Bowden et al. 2011 (Bowden, A., Fleming, R., & Harbison, S. (2011). A method for DNA and RNA co-extraction for use on forensic samples using the Promega DNA IQ™ system. Forensic Science International: Genetics, 5(1), 64-68).

All such methods are intended to be included within the scope of the present invention.

Percent Identity

Variant polynucleotide sequences preferably exhibit at least 70%, more preferably at least 71%, more preferably at least 72%, more preferably at least 73%, more preferably at least 74%, more preferably at least 75%, more preferably at least 76%, more preferably at least 77%, more preferably at least 78%, more preferably at least 79%, more preferably at least 80%, more preferably at least 81%, more preferably at least 82%, more preferably at least 83%, more preferably at least 84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, and most preferably at least 99% identity to a specified polynucleotide sequence. Identity is found over a comparison window of at least 10 nucleotide positions, more preferably at least 10 nucleotide positions, more preferably at least 12 nucleotide positions, more preferably at least 13 nucleotide positions, more preferably at least 14 nucleotide positions, more preferably at least 15 nucleotide positions, more preferably at least 16 nucleotide positions, more preferably at least 17 nucleotide positions, more preferably at least 18 nucleotide positions, more preferably at least 19 nucleotide positions, more preferably at least 20 nucleotide positions, more preferably at least 21 nucleotide positions and most preferably over the entire length of the specified polynucleotide sequence. The invention includes such variants.

Polynucleotide sequence identity can be determined in the following manner. The subject polynucleotide sequence is compared to a candidate polynucleotide sequence using BLASTN (from the BLAST suite of programs, version 2.2.5 [November 2002]) in bl2seq (Tatiana A. Tatusova, Thomas L. Madden (1999), “Blast 2 sequences—a new tool for comparing protein and nucleotide sequences”, FEMS Microbiol Lett. 174:247-250), which is publicly available from NCBI (ftp://ftp.ncbi.nih.gov/blast/). The default parameters of bl2seq are utilized except that filtering of low complexity parts should be turned off.

The identity of polynucleotide sequences may be examined using the following unix command line parameters:

bl2seq -i nucleotideseq1-j nucleotideseq2-F F -p blastn

The parameter -F F turns off filtering of low complexity sections. The parameter -p selects the appropriate algorithm for the pair of sequences. The bl2seq program reports sequence identity as both the number and percentage of identical nucleotides in a line “Identities=”.

Polynucleotide sequence identity may also be calculated over the entire length of the overlap between a candidate and subject polynucleotide sequences using global sequence alignment programs (e.g. Needleman, S. B. and Wunsch, C. D. (1970) J. Mol. Biol. 48, 443-453). A full implementation of the Needleman-Wunsch global alignment algorithm is found in the needle program in the EMBOSS package (Rice, P. Longden, I. and Bleasby, A. EMBOSS: The European Molecular Biology Open Software Suite, Trends in Genetics June 2000, vol 16, No 6. pp. 276-277) which can be obtained from http://www.hgmp.mrc.ac.uk/Software/EMBOSS/. The European Bioinformatics Institute server also provides the facility to perform EMBOSS-needle global alignments between two sequences on line at http:/www.ebi.ac.uk/emboss/align/.

Alternatively the GAP program, which computes an optimal global alignment of two sequences without penalizing terminal gaps, may be used to calculate sequence identity. GAP is described in the following paper: Huang, X. (1994) On Global Sequence Alignment. Computer Applications in the Biosciences 10, 227-235.

Sequence identity may also be calculated by aligning sequences to be compared using Vector NTI version 9.0, which uses a Clustal W algorithm (Thompson et al., 1994, Nucleic Acids Research 24, 4876-4882), then calculating the percentage sequence identity between the aligned sequences using Vector NTI version 9.0 (Sep. 2, 2003 ©1994-2003 InforMax, licensed to Invitrogen).

In general terms therefore the invention provides a method for the detection of an RNA sequence in a sample. The method including the steps of:
a) providing a sample, and
b) detecting the RNA sequence using at least one primer or probe complementary to a stable region of the RNA sequence.

The stable region of the RNA sequence will preferably be identified using RNA sequencing of the sample and, in particular, will be identified as a region in the RNA sequence which has more aligned sequencing reads than another region, or regions, of the same RNA sequence.

Stable regions have been identified and discussed herein and stable regions for use in the methods of the invention can be selected from the group comprising SEQ ID NO:6 to SEQ ID NO:10 and SEQ ID NO:39 to SEQ ID NO:56 or a compliment of anyone thereof.

Primers have also been identified and discussed herein and primers can be selected from the group comprising SEQ ID NO:11 to SEQ ID NO:20 or compliment of anyone thereof.

Probes have also been identified and discussed herein and can be selected from the group comprising SED ID NO:57 to SEQ ID NO:92 or compliment of anyone thereof.

Additionally, in a more specific sense, the invention can be seen to include a nucleotide sequence comprising at least 5 nucleotides with at least 70% identity to a sequence selected from SEQ ID NO:6 to SEQ ID NO:10 or a compliment thereof, or a sequence selected from SEQ ID NO:39 to SEQ ID NO:56 or a compliment thereof.

Further, and again in a more specific sense, the invention can be seen to include a nucleotide sequence comprising at least 5 nucleotides of a sequence selected from SEQ ID NO:6 to SEQ ID NO:10 or a compliment thereof, or a sequence selected from SEQ ID NO:39 to SEQ ID NO:56 or a compliment thereof.

Further, and again in a more specific sense, the invention can be seen to include a nucleotide sequence comprising at least 10 nucleotides with at least 70% identity to a sequence selected from SEQ ID NO:6 to SEQ ID NO:10 or a compliment thereof, or a sequence selected from SEQ ID NO:39 to SEQ ID NO:56 or a compliment thereof.

Further, and again in a more specific sense, the invention can be seen to include a nucleotide sequence comprising at least 10 nucleotides of a sequence selected from SEQ ID NO:6 to SEQ ID NO:10 or a compliment thereof, or a sequence selected from SEQ ID NO:39 to SEQ ID NO:56 or a compliment thereof.

Further, and again in a more specific sense, the invention to be seen to include a nucleotide sequence selected from any one of SEQ ID NO:57 to SEQ ID NO:92

The use of a nucleotide sequence as is defined above in the typing of a sample including RNA specifically forms part of the present invention.

As will be apparent, samples containing RNA can be taken from a variety of sources. The most preferable sample is a biological tissue sample which can be either solid or liquid.

The samples can be from internal body organs from human or nonhuman animals and can be selected from any one or more of the group comprising heart, brain, liver, fat, muscle, gastrointestinal tract, lung and bone.

The method of the present invention is particularly suitable for use in the forensic field and therefore the sample can be a forensic sample of any type containing RNA such as selected from the group comprising blood, buccal, saliva, menstrual blood, skin, semen and vaginal fluid.

The RNA should preferably be extracted from the sample prior to the detecting step and the RNA sequence can be detected directly or indirectly as will be known to a skilled person. It is however referred that the RNA sequence is detected indirectly by detection of a complementary DNA (cDNA) corresponding to the RNA sequence.

The invention, in a more particular sense, can also be seen to include a method of typing a sample including RNA where the method includes the steps of:
a) providing a sample including RNA;
b) detecting one or more stable RNA sequences in the sample using at least one primer or probe complementary to the one or more stable region of the RNA;

wherein the stable RNA sequence is specific for the type of sample; and

wherein detecting the stable RNA sequence indicates the type of sample.

The invention, in another sense, can be seen to include a method of typing a sample including degraded RNA, the method including the steps:
a) providing a sample including degraded RNA;
b) detecting one or more stable RNA sequences in the sample using at least one primer or probe complementary to the one or more stable region of the degraded RNA;

wherein the stable RNA sequence is specific for the type of sample; and

wherein detecting the target RNA sequence indicates the type of sample.

In another embodiment the invention can be a method for the identification of a stable region in RNA in a sample, the method comprising:
a) providing a sample including RNA,
b) isolating total RNA from the sample,
c) removing DNA from the sample
d) generating cDNA complementary to the RNA in the sample,
e) sequencing the cDNA.

wherein the stable region of the RNA sequence is identified as a region in the RNA sequence which has more aligned sequencing reads than another region, or regions, of the same RNA sequence.

As has been previously discussed, the method can be applied to RNA which has degraded to a condition which had previously been thought not to be useful as a means for typing/identifying the source of the sample from which it has been extracted. The methods of the invention can be used to type/identify the source of samples in which the RNA content has a RIN value of less than 8. As stable regions in RNA having a value of less than eight will also be present in RNA having a RIN value of between 8 and 10, once the stable regions have been identified those stable regions can also be used to identify/type the source of the sample having an RIN of between 8 and 10. Therefore, the method can be used to type/identify the source of samples having any RIN value, including samples in which the RIN value cannot be determined.

As has been discussed previously, the stable region of the RNA sequence can be identified as a region in the RNA sequence which has more aligned sequencing reads than another region, or regions, of the same RNA sequence.

As will be readily apparent to a skilled person, the RNA sequence will preferably be detected using a primer or a probe. As will also be apparent, the RNA sequence can be detected using more than one primer or probe (e.g. two primers) if appropriate/desired.

The primers and should preferably correspond to, or be complementary to, or be capable of hybridising to, a sequence within the stable region of the RNA that has been extracted from the sample. The primers are used to amplify the part of the stable region bound by the primers, such as by a polymerase chain reaction (PCR) method. The PCR method can be selected from standard PCR, reverse transcriptase (RT)-PCR, and quantitative reverse transcriptase PCR (qRT-PCR).

In addition, and as will also be readily apparent to a skilled person, the RNA sequence can be detected using a probe. This will preferably correspond to, or be complementary to, a sequence within the stable region of the RNA that has been extracted from the sample.

As has been discussed previously, the samples to be typed/identified containing the RNA can be taken from a variety of sources. While forensic samples (e.g. body tissues of variety of types) are of particular interest, the samples can also be taken from an environmental or processing source. For example, the method can be used for the detection of invasive species for example, in biosecurity testing. Field samples can be taken and identified from plant (partial leaf, cuttings, sap/exudate or root material), animal (biological fluid/biopsy), human (biological fluid/biopsy) and marine/aquaculture material (marine animals, fish, plant, algae and water quality). The non-pristine nature and limited abundance of field samples make the detection of target RNA from invasive species (virus and other microorganisms) difficult due to limits of detection sensitivity, subsequently limiting specificity.

The RNA sequence can be encoded by a marker gene specific for the type of sample. That is, the expression of the RNA sequence, or presence of the RNA sequence, in the sample, is diagnostic for the type of sample. For example, when the sample is circulatory blood, the marker gene can be selected from:
Hemoglobin delta (HBD),
Solute carrier family 4 (anion exchanger), member 1 (Diego blood group) (SLC4A1),
Glycophorin A (MNS blood group) (GYPA),
Hemoglobin, beta (HBB), and
Pro-platelet basic protein (chemokine (C-X-C motif) ligand 7) (PPBP).

Further, when the sample is oral mucosa/saliva (buccal), the marker genes can be selected from:
the saliva marker Histatin 3 (HTN3),
Proline-rich protein BstNI subfamily 4 (PRB4), and
Statherin (STATH)

Further, when the sample is menstrual blood, the marker genes can be selected from:
Matrix metallopeptidase 11 (MMP11),
Matrix metallopeptidase 10 (stromelysin 2) (MMP10),
Matrix metallopeptidase 3 (MMP3),
Matrix metallopeptidase 7 (MMP7), and
Stanniocalcin 1 (STC1).

Further, when the sample is vaginal fluid, the marker genes is Chemokine (C-X-C motif) ligand 8 (CXCL8).

The detection process can involve the use of either a primer or a probe capable of hybridising to the stable region of the RNA sequence, or a cDNA corresponding to the stable region or a complement thereof. The method may involve using just one pair of primers, or a single probe, to type the sample. Alternatively multiple pairs of primers, or multiple probes, may be used.

The primer or the probe can include (i) a sequence of at least 5 nucleotides with at least 70% identity to any part of the sequence of any one of SEQ ID NO: 6 to 10 and 39 to 56 or a complement thereof or (ii) a sequence of at least 5 nucleotides with at least 70% identity to the sequence of any one of SEQ ID NO: 6 to 10 and 39 to 56, or a complement thereof or (iii) a sequence of at least 5 nucleotides of the sequence of any one of SEQ ID NO: 6 to 10 and 39 to 56, or a complement thereof or (iv) a sequence of at least 5 nucleotides of the sequence of any one of SEQ ID NO: 6 to 10 and 39 to 56, or a complement thereof or (v) a sequence selected from any one of SEQ ID NO: 11 to 20 or (vi) a sequence selected from any one of SEQ ID NO: 11 to 20 or (vii) a label or tag attached to a sequence selected from any one of those sequences and in particular SEQ ID NO: 11 to 20.

The primer or the probe can include (i) a sequence of at least 10 nucleotides with at least 70% identity to any part of the sequence of any one of SEQ ID NO: 6 to 10 and 39 to 56 or a complement thereof or (ii) a sequence of at least 10 nucleotides with at least 70% identity to the sequence of any one of SEQ ID NO: 6 to 10 and 39 to 56, or a complement thereof or (iii) a sequence of at least 10 nucleotides of the sequence of any one of SEQ ID NO: 6 to 10 and 39 to 56, or a complement thereof or (iv) a sequence of at least 10 nucleotides of the sequence of any one of SEQ ID NO: 6 to 10 and 39 to 56, or a complement thereof or (v) a sequence selected from any one of SEQ ID NO: 57 to 92 or (vi) a sequence selected from any one of SEQ ID NO: 57 to 92 or (vii) a label or tag attached to a sequence selected from any one of those sequences and in particular SEQ ID NO: 57 to 92.

By way of example, typing of a sample can be undertaken using multiplex PCR performed with multiple primers, at least one of which is diagnostic for the type of sample.

Preferably multiplex PCR is performed using at least 4, more preferably at least 5, more preferably at least 6, more preferably at least 7, more preferably at least 8, more preferably at least 9, more preferably at least 10, more preferably at least 11, more preferably at least 12, more preferably at least 13, more preferably at least 14, more preferably at least 15, more preferably at least 16, more preferably at least 17, more preferably at least 18, more preferably at least 19, more preferably at least 20, more preferably at least 21, more preferably at least 22, more preferably at least 23, more preferably at least 24, more preferably at least 25, more preferably at least 26, more preferably at least 27, more preferably at least 28, more preferably at least 29, more preferably at least 30 primers of the invention.

The invention also allows the provision of a kit that includes at least one primer or probe according to the present invention. Such a kit can include any number of primers or probes and in particular the kit can include at least 2, more preferably at least 3, more preferably at least 4, more preferably at least 5, more preferably at least 6, more preferably at least 7, more preferably at least 8, more preferably at least 9, more preferably at least 10, more preferably at least 11, more preferably at least 12, more preferably at least 13, more preferably at least 14, more preferably at least 15, more preferably at least 16, more preferably at least 17, more preferably at least 18, more preferably at least 19, more preferably at least 20, more preferably at least 21, more preferably at least 22, more preferably at least 23, more preferably at least 24, more preferably at least 25, more preferably at least 26, more preferably at least 27, more preferably at least 28, more preferably at least 29, more preferably at least 30 primers or probes of the invention. Combinations of primers and probes may also be provided in such kits.

As will be readily apparent, the kit should also include instructions for use, if such instructions are needed.

The invention also allows the provision of microarrays or chips or like products that include sequences that have been identified herein as stable areas of RNA that can be used to type/identify samples or that are complimentary thereto. These sequences have been used to generate primers and probes that can be used on microarrays or chips or like products for the detection of nucleotide sequences.

Such microarrays or chips are of particular commercial importance as they allow the efficient and accurate identification of unknown samples including RNA, including where the RNA has been degraded. The creation of such products as well within the abilities of the person skilled in the art once they have the benefit of knowledge of the present invention.

### BRIEF DESCRIPTION OF DRAWINGS

FIG. 1. (A) Sequencing reads from 6 week old male buccal samples, aligned to the reference genome hg19 and viewed in the sequence viewing software Geneious v5.5. The black features depict the position of RT-PCR forward and reverse primers for amplification of the saliva marker HTN3 (NM_000200.2) [31-34], designed using conventional primer design methodology, without consideration for RNA stability. X denotes level of sequencing read coverage along the reference; Y denotes the annotated reference gene; Z denotes the alignment of sequencing reads along the reference. (B) Sequencing reads from 6 week old male buccal samples, aligned the reference genome hg19 and viewed in the sequence viewing software Geneious v5.5. The white features depict the position of RT-PCR forward and reverse primers for amplification of the saliva marker HTN3, designed using the new approach, with priority given to targeting RNA regions of high sequencing read coverage (higher RNA stability). X denotes level of sequencing read coverage along the reference; Y denotes the annotated reference gene; Z denotes the alignment of sequencing reads along the reference. (C) Electropherogram of a singlex PCR amplification of cDNA from 1 week old buccal samples using conventionally designed HTN3 primers (orange arrow) and HTN3 primers designed to target the stable RNA region (pink).

FIG. 2. (A) Sequencing reads from 6 week old male circulatory blood samples, aligned to the reference genome hg19 and viewed in the sequence viewing software Geneious v5.5. The black features depict the position of RT-PCR forward and reverse primers for amplification of the housekeeping gene UBE2D2 (NM_003339.2) [31, 32], designed using conventional primer design methodology, without consideration for RNA stability. The white features depict the position of RT-PCR forward and reverse primers for amplification of the housekeeping gene UBE2D2, designed using the new approach, with priority to targeting RNA regions of high sequencing read coverage (higher RNA stability). X denotes level of sequencing read coverage along the reference; Y denotes the annotated reference gene; Z denotes the alignment of sequencing reads along the reference. (B) Electropherogram of a singlex PCR amplification of cDNA from one month old circulatory blood using conventionally designed UBE2D2 primers (black arrow) and UBE2D2 primers designed to target the stable RNA region (white arrow).

FIG. 3. (A) Sequencing reads from 6 week old female circulatory blood samples, aligned to the reference genome hg19 and viewed in the sequence viewing software Geneious v5.5. The black features depict the position of RT-PCR forward and reverse primers for amplification of a common blood marker, HBD (NM_000519), designed using conventional primer design methodology, without consideration for RNA stability. The white features depict the position of RT-PCR forward and reverse primers for amplification of a common blood marker, HBD, designed using the new approach, with priority given to targeting RNA regions of high sequencing read coverage (higher RNA stability). X denotes level of sequencing read coverage along the reference; Y denotes the annotated reference gene; Z denotes the alignment of sequencing reads along the reference. (B) Relative fluorescent units detected from singlex PCR amplifications of cDNA from various body fluids (BA2=16 day old circulatory blood; BH1=19 day old circulatory blood; MA4=13 day old menstrual blood; MD2=1 week old menstrual blood) using conventionally designed HBD primers (black) and HBD primers designed to target the stable RNA region (white).

FIG. 4. (A) Sequencing reads from 6 week old male circulatory blood samples, aligned to the reference genome hg19 and viewed in the sequence viewing software Geneious v5.5. The black features depict the position of RT-PCR forward and reverse primers for amplification of SLC4A1 (NM_000342.3), designed using conventional primer design methodology, without consideration for RNA stability. The white features depict the position of RT-PCR forward and reverse primers for amplification of SLC4A1, designed using the new approach, with priority given to targeting RNA regions of high sequencing read coverage (higher RNA stability). X denotes level of sequencing read coverage along the reference; Y denotes the annotated reference gene; Z denotes the alignment of sequencing reads along the reference. (B) Relative fluorescent units detected from singlex PCR amplifications of cDNA from various body fluids (BA2=16 day old circulatory blood; BH1=19 day old circulatory blood; MA4=13 day old menstrual blood; MD2=1 week old menstrual blood) using conventionally designed SLC4A1 primers (black) and SLC4A1 primers designed to target the stable RNA region (white).

FIG. 5. (A) Sequencing reads from 6 week old menstrual blood samples, aligned to the reference genome hg19 and viewed in the sequence viewing software Geneious v5.5. The black features depict the position of RT-PCR forward and reverse primers for amplification of the menstrual blood marker, MMP11 (NM_005940.3) [31, 33], designed using conventional primer design methodology, without consideration for RNA stability. The white features depict the position of RT-PCR forward and reverse primers for amplification of MMP11, designed deliberately for a region of lower RNA stability. X denotes level of sequencing read coverage along the reference; Y denotes the annotated reference gene; Z denotes the alignment of sequencing reads along the reference. (B) Electropherogram of a singlex PCR amplification of cDNA from one day old menstrual blood using conventionally designed MMP11 primers (black arrow) and MMP11 primers designed deliberately to target a region of lower RNA stability (white arrow). (C) Electropherogram of a singlex PCR amplification of cDNA from 6 week old menstrual blood using conventionally designed MMP11 primers (black arrow) and MMP11 primers designed to target a region of lower RNA stability (white arrow).

The invention will now be exemplified by way of the following non-limiting examples.

### EXAMPLE

### Example 1: Use of the Method of the Invention to Detect RNA Sequences in Degraded Samples

Materials and Methods

Body Fluid Sampling and Ageing (RNA Degradation)

Fresh body fluid samples (oral mucosa/saliva (buccal), circulatory blood, vaginal fluid and menstrual blood) were collected on sterile Cultiplast® rayon swabs and aged at room temperature with exposure to ambient laboratory conditions, for t=0, two and six weeks. Samples were collected from two individuals for circulatory blood and buccal and from one individual for menstrual blood and vaginal fluid. Triplicate samples (2 swabs per replicate) were collected on the same day from each individual, for each body fluid at each time point. Oral mucosa/saliva, vaginal fluid and menstrual blood samples were obtained by swabbing by the participants themselves while 50 μL of fresh circulatory blood was drawn using a sterile ACCU-CHEK® Safe-T-Pro Plus lancet (Roche Diagnostics USA, Indianapolis, Ind., USA) and deposited onto each swab.

RNA Extraction

Total RNA for all samples was extracted using the Promega® ReliaPrep™ RNA Cell Miniprep System (Promega Corporation, Madison, Wis., USA) following the manufacturer's instructions. DNA was removed from extracted RNA using on-column DNase I treatment during the RNA extraction process. RNA was eluted in 50 uL elution buffer. Complete removal of human DNA was verified using the Quantifiler® Human DNA quantification kit (Life Technologies Corp., Carlsbad, Calif., USA) using 1 uL of sample in a 12.5 uL reaction.

Library Preparation and Sequencing

cDNA libraries for RNAseq were prepared using Bioo Scientific NEXTFlex directional RNA-seq Kit (dUTP-Based) v2 48 (Bioo Scientific, Austin, Tex., USA). Total RNA was not subjected to ribosomal RNA depletion. Due to the low concentration and degraded nature of some samples, 13 μl total RNA input was used for library preparation irrespective of concentration. One microlitre of ERCC controls (Life Technologies Corp., Carlsbad, Calif., USA) diluted 1000 fold was added to each sample. Barcodes (1-16) were added to each library using the NEXTflex RNA-Seq barcodes kit (Bioo Scientific, Austin, Tex., USA).

Barcoded libraries were sequenced across three lanes on an Illumina HiSeq2500 sequencer, with 2×100 bp paired-end chemistry.

Bioinformatics Analysis

Read quality for all samples were analysed using SolexaQA [35]. Data was preprocessed using DynamicTrim v1.9 using default settings [35]. Data was length-sorted and unpaired reads discarded using Lengthsort v1.9 using default settings [35]. Subsequent processed data consisted entirely of reads with <5% probability of error (or a Q score of >13), with pairs, and length >25 bp.

Reads were aligned to the human genome hg19 (GRCh37) [36]. The “UCSC genes” annotation track of known genes was downloaded from the UCSC genome browser as hg19_UCSC_genes.gtf [36].

FASTA and gtf format files of External RNA Controls Consortium (ERCC) spike-in controls were obtained from the manufacturer's website (http://www.lifetechnologies.com/order/catalog/product/4456739). These ERCC annotations were concatenated onto the end of the hg19 FASTA and gtf annotation tracks. ERCC controls were analysed in the same way as the other genes in subsequent analyses.

Processed reads were mapped to the combined human genome (hg19)/ERCC controls using Tophat2 v2.0.12 [37] with the following switches: --library-type fr-firststrand -M $leftread $rightread

Transcripts were reconstructed from splice-aware mapping results from Tophat2, using Cufflinks v2.2.1 [38] with the following switches: -g -b -u --library-type fr-firststrand --library-norm-method geometric

The reconstructed transcripts from each sample were merged into a single .gtf file using Cuffmerge v2.2.1 [39] with the following switches: -g -s

Library size normalised expression (FPKM) for each sample was generated using Cuffnorm v2.2.1 [38] with the following switches: --library-type fr-firststrand -library-norm-method geometric -output-format cuffdiff

cDNA Synthesis

cDNA was synthesised from 10 μL DNA-free RNA from each body fluid sample using random hexamers and the Superscript® III First-Strand Synthesis SuperMix kit (Life Technologies Corp., Carlsbad, Calif., USA).

Primer Design

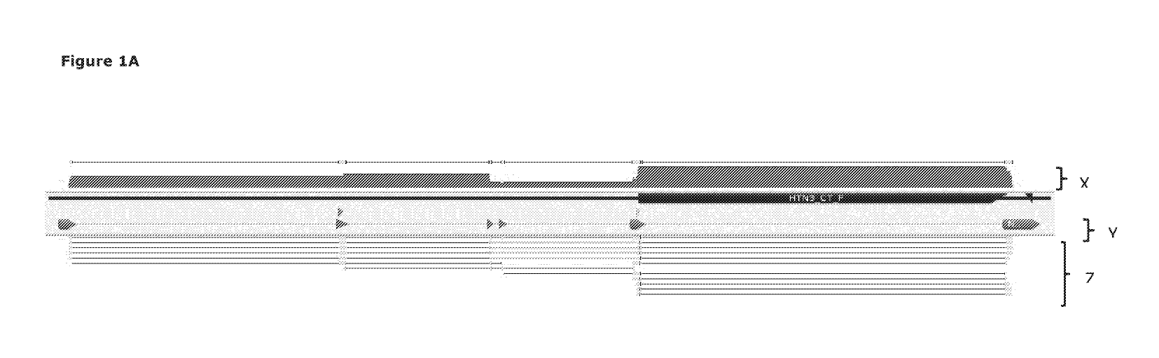

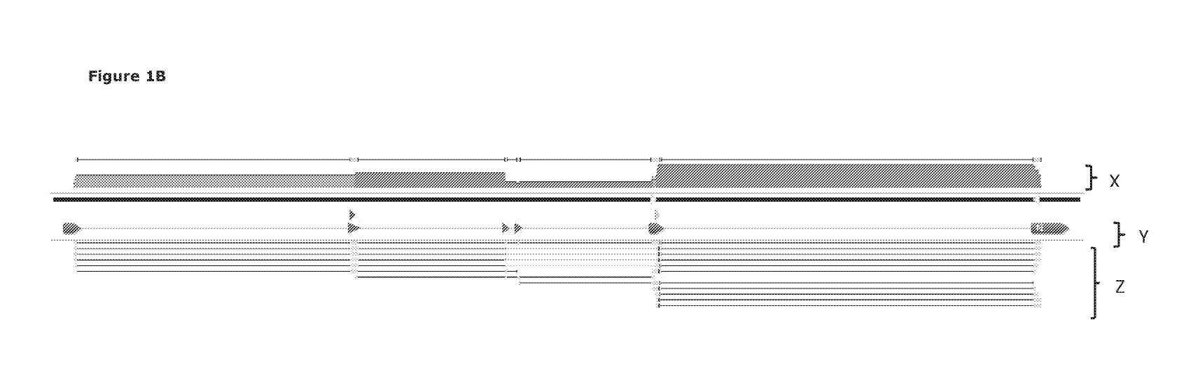

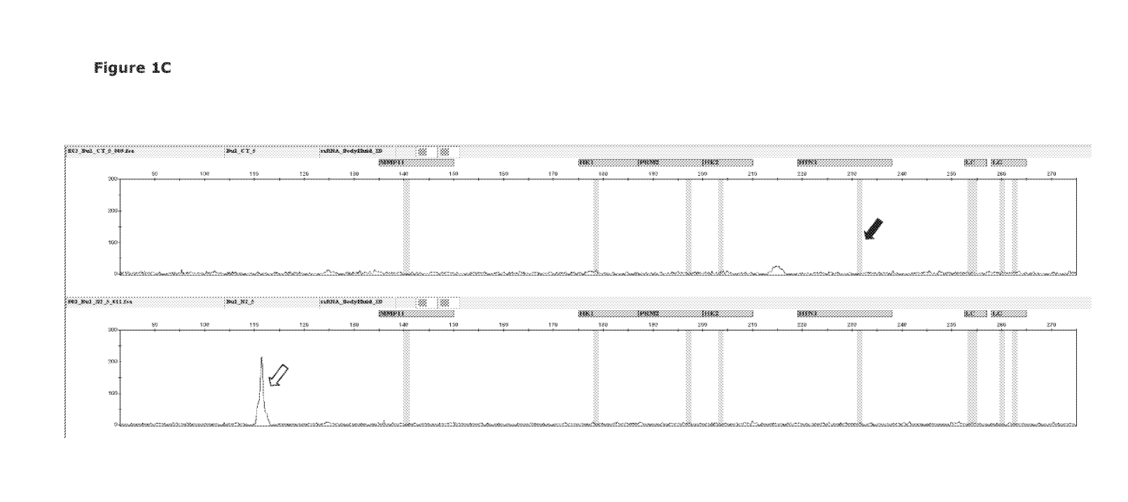

Sequencing read alignments to the reference genome hg19 were viewed using the sequence viewing software Geneious v5.6.7 (Biomatters Ltd, Auckland, New Zealand). Read alignments to particular genes of interest were observed (FIGS. 1a, 1b, 2a, 3a, 4a, 5a) and primers designed using conventional methodology [3-7, 40] were mapped to these alignments (FIGS. 1a, 2a, 3a, 4a, 5a). New primers for the same genes of interest were designed to amplify RNA regions of high sequencing read coverage, deemed to be RNA regions of higher stability (FIGS. 1b, 2a, 3a, 4a, 5a). Importantly, primers designed to target stable RNA regions also conformed to the thermodynamic standards of conventional PCR primer design [3-7, 40].

PCR Amplification

cDNA from body fluid samples were amplified using the Qiagen Multiplex PCR kit (Qiagen GmbH, Hilden, Germany). The PCR primer concentrations, template cDNA and annealing temperatures are detailed in Table 3.

TABLE 3
RNA marker primer and amplification conditions

| | | | Final | Annealing | |
|---|---|---|---|---|---|
| | Primer | Body fluid | concentration | temperature | Input cDNA |
| Marker | target type | specificity | (μM) | (° C.) | (μL) |
| HTN3 | conventional | Buccal/saliva | 0.25 | 58 | 2 |
| F/R | | | | | |
| HTN3 | stable | Buccal/saliva | 0.25 | 58 | 2 |
| F/R | | | | | |
| UBE2D2 | conventional | housekeeping | 0.0125 | 58 | 2 |
| F/R | | | | | |
| UBE2D2 | stable | housekeeping | 0.0125 | 58 | 2 |
| F/R | | | | | |
| HBD F/R | conventional | blood | 0.1 | 65 | 2 |
| HBD F/R | stable | blood | 0.1 | 65 | 2 |
| SLC4A1 | conventional | blood | 0.1 | 65 | 2 |
| F/R | | | | | |
| SLC4A1 | stable | blood | 0.1 | 65 | 2 |
| F/R | | | | | |
| MMP11 | conventional | menstrual | 0.1 | 58 | 2 |
| F/R | | blood | | | |
| MMP11 | degraded | menstrual | 0.1 | 58 | 2 |
| F/R | | blood | | | |

The following PCR program was used:

1) Initial denaturation for 15 mins @ 95° C.;

2) Denaturation for 30 s @ 94° C.;

3) Annealing 3 mins @ appropriate annealing temperature (Table 1);

1) to 3) is repeated for 35 cycles

4) Extension for 1 min @ 72° C.;

5) 45 mins @ 72° C.;

6) 4° C. indefinitely.

Results

HTN3 Conventional Primers Vs HTN3 Primers for Stable Regions

cDNA from 6 week old male buccal samples were amplified using primers for the saliva marker Histatin 3 (HTN3)(NM_000200.2) [31-34], designed using conventional primer design methodology and primers targeting the highly stable RNA region (FIG. 1A-B). PCR amplification using conventional HTN3 primers did not generate a detectable amplicon (FIG. 1C). PCR amplification using the same sample and conditions with new primers to target the stable RNA region generated an amplicon of ˜220 relative fluorescent units (RFU).

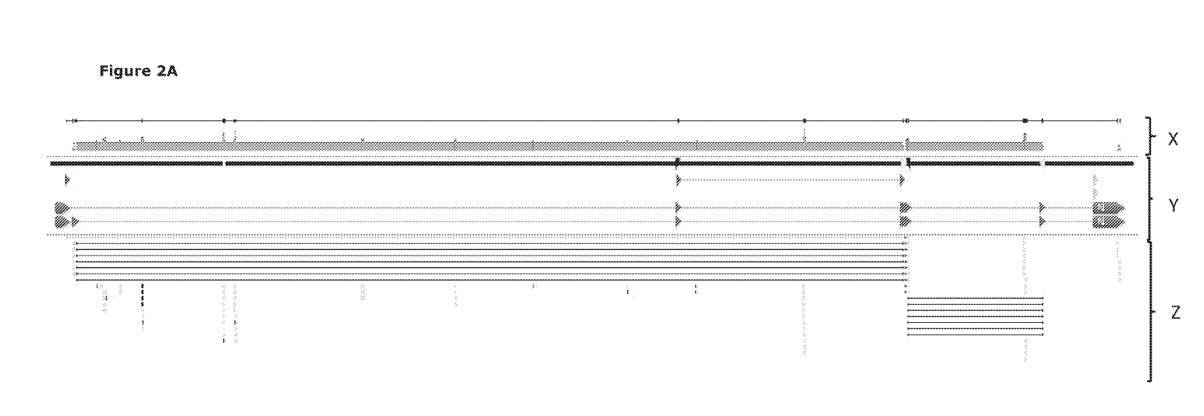

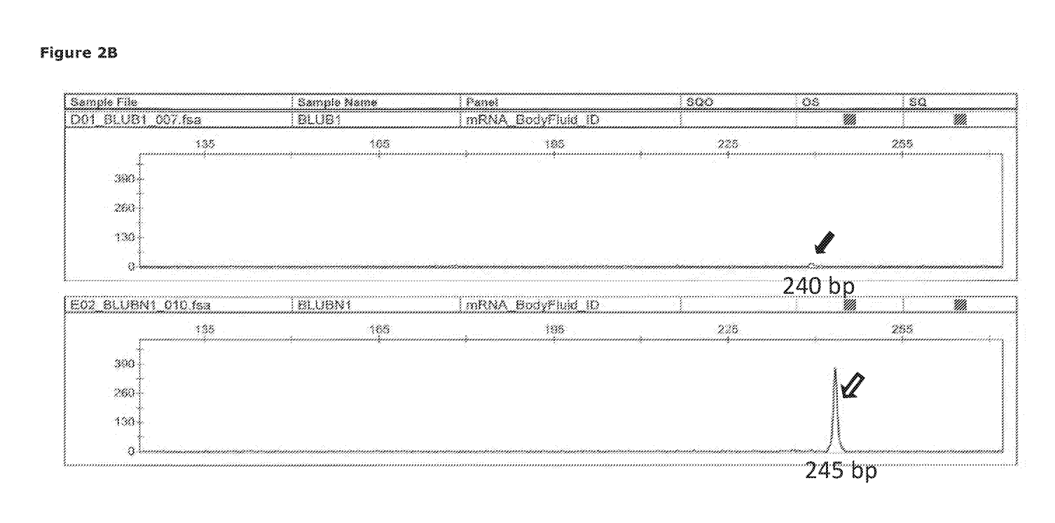

UBE2D2 conventional primers vs UBE2D2 primers for stable regions cDNA from 6 week old male circulatory blood was amplified using primers for the housekeeping gene Ubiquitin-conjugating enzyme E2D 2 (UBE2D2)(NM_003339.2) [31, 32], designed using conventional primer design methodology and primers targeting the highly stable RNA region (FIG. 2A). PCR amplification using conventional UBE2D2 primers generated no detectable amplicon (orange arrow, FIG. 2B). PCR amplification using the same sample and conditions with new primers to target the stable RNA region generated an amplicon of ˜280 RFU (FIG. 2B).

HBD Conventional Primers Vs HBD Primers for Stable Regions

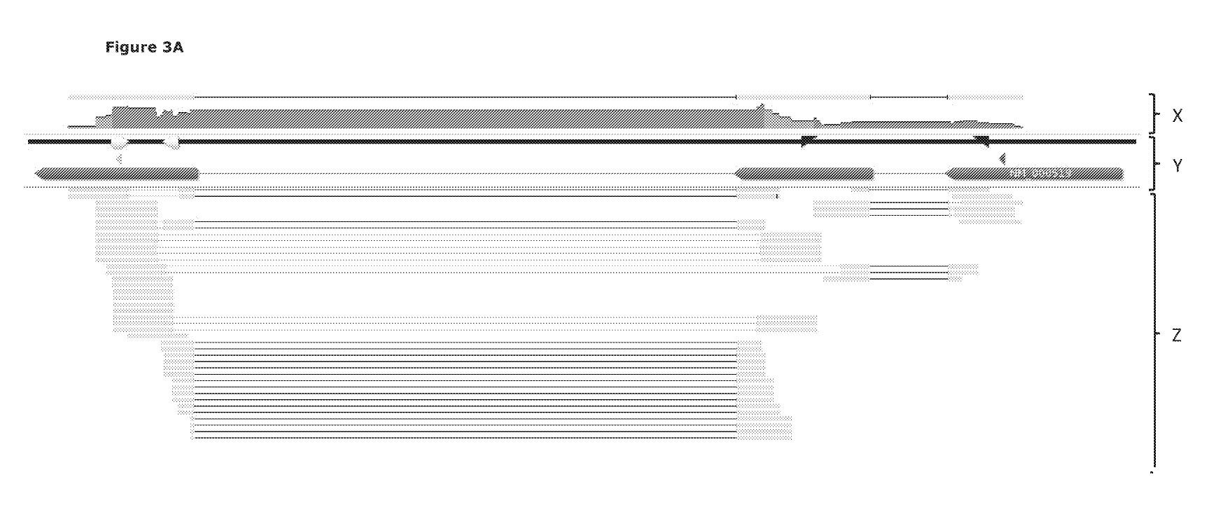

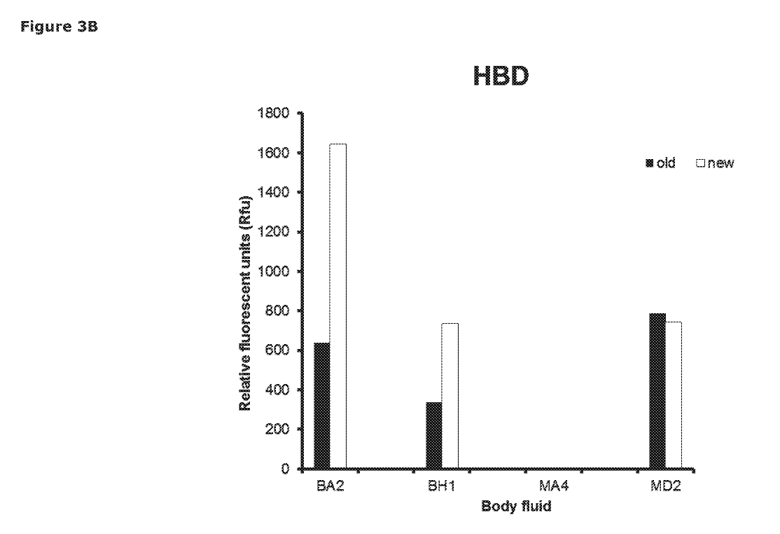

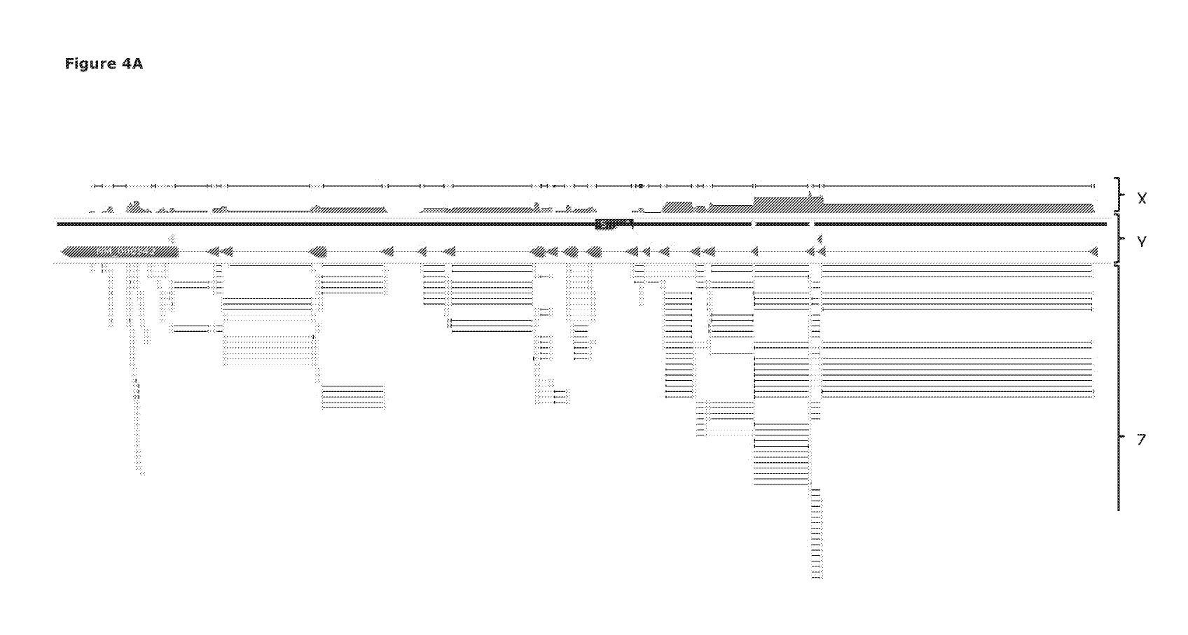

cDNA from 16 day old circulatory blood (BA2), 19 day old circulatory blood (BH1), 13 day old menstrual blood (MA4) and 1 week old menstrual blood (MD2) were amplified using primers for the common blood marker, Hemoglobin, delta (HBD)(NM_000519), designed using conventional primer design methodology and primers targeting the highly stable RNA region (FIG. 3A). PCR amplification of sample BA2 generated an amplicon of just over 600 RFU (FIG. 3B) using conventional HBD primers and an amplicon of just over 1600 RFU using new primers to target the stable RNA region (FIG. 3B). PCR amplification of sample BH1 generated an amplicon of ˜320 RFU (FIG. 3B) using conventional HBD primers and an amplicon of ˜720 RFU using new primers to target the stable RNA region (FIG. 3B). PCR amplification of sample MA4 generated no detectable amplicon (FIG. 3B) using either conventional HBD primers or new primers to target the stable RNA region (FIG. 3B). PCR amplification of sample MD2 generated amplicons of just under 800 RFU (FIG. 3B) using both the conventional HBD primers and using new primers to target the stable RNA region (FIG. 3B).

SLC4A1 Conventional Primers Vs SLC4A1 Primers for Stable Regions

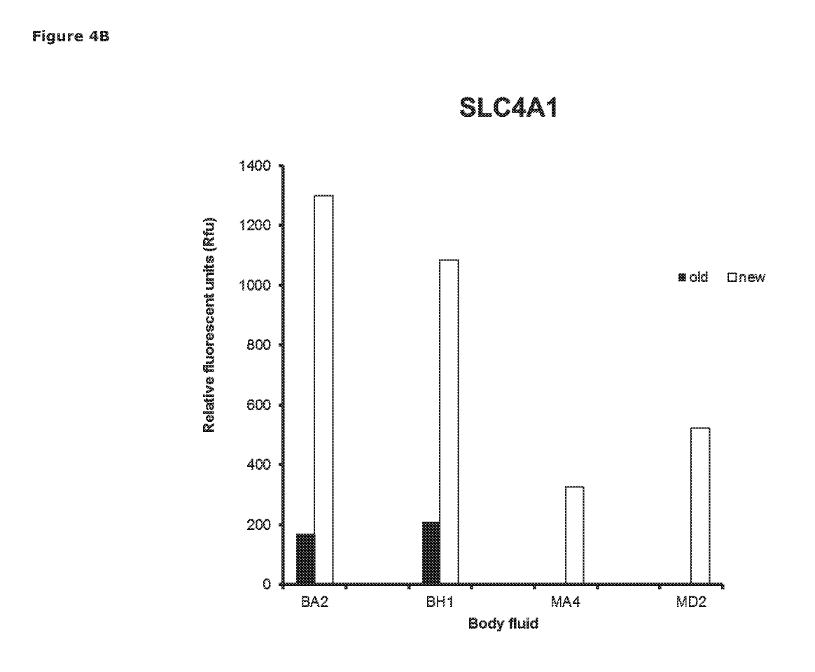

cDNA from 16 day old circulatory blood (BA2), 19 day old circulatory blood (BH1), 13 day old menstrual blood (MA4) and 1 week old menstrual blood (MD2) were amplified using primers for a blood marker, Solute carrier family 4 (anion exchanger), member 1 (Diego blood group) (SLC4A1)(NM_000342.3), designed using conventional primer design methodology and primers targeting the highly stable RNA region (FIG. 4A). PCR amplification of sample BA2 generated an amplicon of ˜180 RFU (FIG. 4B) using conventional SLC4A1 primers and an amplicon of just over ˜1300 RFU using new primers to target the stable RNA region (FIG. 4B). PCR amplification of sample BH1 generated an amplicon of just over 200 RFU (FIG. 4B) using conventional SLC4A1 primers and an amplicon of ˜1100 RFU using new primers to target the stable RNA region (FIG. 4B). PCR amplification of sample MA4 generated no detectable amplicon (FIG. 4B) using conventional SLC4A1 primers and an amplicon of ˜350 RFU using new primers to target the stable transcript region (FIG. 4B). PCR amplification of sample MD2 generated no detectable amplicon (FIG. 4B) using conventional SLC4A1 primers and an amplicon of ˜500 RFU using new primers to target the stable RNA region (FIG. 4B).

MMP11 Conventional Primers Vs MMP11 Primers for Degraded Regions

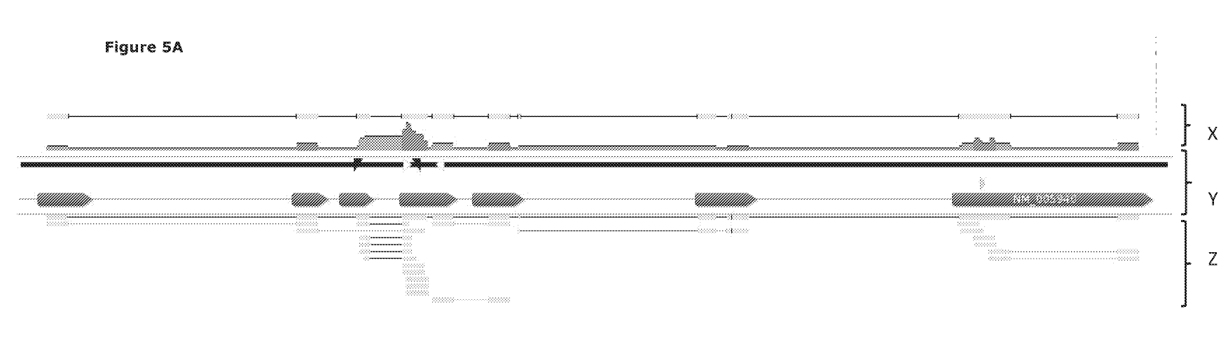

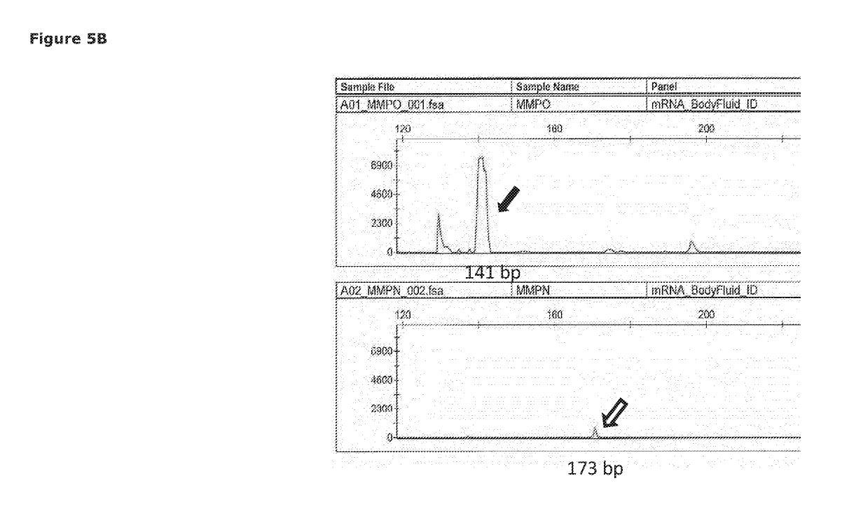

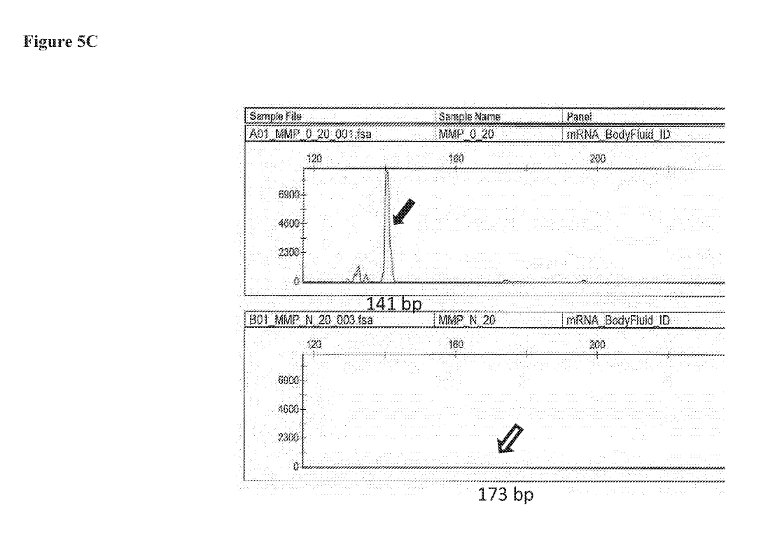

cDNA from 1 day old menstrual blood and 6 week old menstrual blood was amplified using primers for the menstrual blood marker Matrix metallopeptidase 11 (MMP11) (NM_005940.3) [31, 33], designed using conventional primer design methodology and primers to deliberately target a degraded RNA region (FIG. 5A). PCR amplification of 1 day old menstrual blood generated an amplicon of ˜8000 RFU (FIG. 5B) using conventional MMP11 primers and an amplicon of just over ˜1000 RFU using new primers to target a degraded RNA region (FIG. 5B). PCR amplification of 6 week old menstrual blood generated an amplicon of ˜9000 RFU (FIG. 5C) using conventional MMP11 primers and no detectable amplicon using new primers to target a degraded RNA region (FIG. 5C).

### Examples 2 and 3

Examples 2 and 3 below show RNA integrity (RIN) scores of samples typed using primers corresponding to stable regions that have been identified according to the invention, and RIN scores of samples used for stable region identification. As is shown, the methods of the invention are useful for samples having a range of RIN scores, including RIN scores of less than 8 and also where RIN is undetermined (beyond detection).

Body Fluid Sampling and Ageing (RNA Degradation)

Fresh body fluid samples (oral mucosa/saliva (buccal), circulatory blood, vaginal fluid and menstrual blood) were collected on sterile Cutiplast® rayon swabs (n=6) and aged at room temperature with exposure to ambient laboratory conditions (including sunlight), for t=0, two and six weeks. Oral mucosa/saliva, vaginal fluid and menstrual blood samples were obtained by swabbing by the participants themselves while 50 μL of fresh circulatory blood drawn using a sterile ACCU-CHEK® Safe-T-Pro Plus lancet (Roche Diagnostics USA, Indianapolis, Ind., USA)—was deposited onto each swab.

RNA Extraction

Total RNA for all samples was extracted using the Promega® ReliaPrep™ RNA Cell Miniprep System (Promega Corporation, Madison, Wis., USA) following the manufacturer's instructions. DNA was removed from extracted RNA using on-column DNase I treatment during the RNA extraction process. RNA was eluted in 50 uL elution buffer. Complete removal of human DNA was verified using the Quantifiler® Human DNA quantification kit (Life Technologies Corp., Carlsbad, Calif., USA) using 1 uL of sample in a 12.5 uL reaction.

RNA Integrity Analysis and Quantification

RNA integrity for each sample was determined using the Agilent RNA 6000 pico kit (Agilent Technologies, Santa Clara, Calif., USA) and the 2100 Bioanalyzer instrument (Agilent Technologies, Santa Clara, Calif., USA).

### Example 2

RNA Integrity (RIN) Scores of Samples Typed Using Primers Based on Stable Regions

| | Degradation | | |
|---|---|---|---|
| Sample | time (days) | Degradation conditions | RIN score |
| circulatory blood | 0 | ambient laboratory | 8.2 |
| circulatory blood | 42 | ambient laboratory | 2.8 |
| circulatory blood | 16 | ambient laboratory | 1 |
| | | overnight; −20° C. | |
| | | thereafter | |
| circulatory blood | 19 | ambient laboratory | undetermined |
| | | overnight; −20° C. | |
| | | thereafter | |
| oral mucosa/ | 0 | ambient laboratory | 2.3 |
| saliva (buccal) | | | |
| oral mucosa/ | 42 | ambient laboratory | 1 |
| saliva (buccal) | | | |
| menstrual blood | 0 | ambient laboratory | 4.4 |
| menstrual blood | 42 | ambient laboratory | undetermined |
| menstrual blood | 13 | ambient laboratory | undetermined |
| | | overnight; −20° C. | |
| | | thereafter | |
| menstrual blood | 7 | ambient laboratory | undetermined |
| | | overnight; −20° C. | |
| | | thereafter | |

### Example 3

RNA Integrity (RIN) Scores of Samples Used for Stable Region Identification Using Next Generation Sequencing (NGS)

| | Degradation time | RIN |
|---|---|---|
| Body fluid | (weeks) | score |
| oral mucosa/saliva (buccal) | 0 | 1.9 |
| oral mucosa/saliva (buccal) | 0 | 1.9 |
| oral mucosa/saliva (buccal) | 0 | 1.8 |
| oral mucosa/saliva (buccal) | 6 | 2.1 |
| oral mucosa/saliva (buccal) | 6 | 2.3 |
| oral mucosa/saliva (buccal) | 6 | 2.3 |
| oral mucosa/saliva (buccal) | 0 | 2.5 |
| oral mucosa/saliva (buccal) | 0 | 2.6 |
| oral mucosa/saliva (buccal) | 0 | 3 |
| oral mucosa/saliva (buccal) | 6 | 1 |
| oral mucosa/saliva (buccal) | 6 | 1 |
| oral mucosa/saliva (buccal) | 6 | 1 |
| vaginal fluid | 0 | 3.6 |
| vaginal fluid | 0 | 2.6 |
| vaginal fluid | 0 | 2.6 |
| vaginal fluid | 2 | 2.4 |
| vaginal fluid | 2 | 2.4 |
| vaginal fluid | 2 | 2.4 |
| vaginal fluid | 6 | 2.4 |
| vaginal fluid | 6 | 2.4 |
| vaginal fluid | 6 | 2.5 |
| circulatory blood | 0 | 7.6 |
| circulatory blood | 0 | 7.7 |
| circulatory blood | 0 | 8.2 |
| circulatory blood | 2 | 5.1 |
| circulatory blood | 2 | 5.1 |
| circulatory blood | 6 | 2.4 |
| circulatory blood | 6 | 2.8 |
| circulatory blood | 6 | 2.8 |
| circulatory blood | 0 | 7.6 |
| circulatory blood | 2 | 8 |
| circulatory blood | 0 | 7.8 |
| circulatory blood | 2 | 5.4 |
| circulatory blood | 2 | 5.1 |
| circulatory blood | 2 | 5.8 |
| circulatory blood | 6 | 3.6 |
| circulatory blood | 6 | 3.9 |
| circulatory blood | 6 | 4.1 |
| menstrual blood | 0 | 4.4 |
| menstrual blood | 0 | 3.9 |
| menstrual blood | 0 | 5.4 |
| menstrual blood | 2 | 2.1 |
| menstrual blood | 2 | 2.2 |
| menstrual blood | 2 | 2.2 |
| menstrual blood | 6 | 3.8 |
| menstrual blood | 6 | N/A |
| menstrual blood | 6 | N/A |

### GENERAL

The above Examples show that the methods and materials of the invention can be used to type samples at varying levels of degradation as indicated by their RIN values. The Examples clearly demonstrate the ability of the of the methods and materials of the invention to type samples having RIN values of less than 8, which is in contrast to commonly held view. The ability to identify stable areas of RNA that can be used to type samples has clearly been demonstrated, and has been demonstrated at a variety of RIN values. In particular, it is notable that the use of primers according to the invention which target the highly stable RNA regions improve detection accuracy when compared to conventional primers.

The ability to prepare microarrays which include primers according to the invention (which target the stable RNA regions) allows accurate and efficient typing of unknown samples to be completed in circumstances where this has previously been difficult if not impossible.

As has been discussed previously within the specification, this invention has particular application within the forensic science field where samples have usually been degraded over time in the environment that the samples are in, or as a result of temperature, pressure, or other processing conditions. The ability to type such samples is of clear advantage to the users as it allows typing of samples from real time circumstances and conditions. This was previously not considered to be an option prior to the present invention.

The foregoing describes the invention including known variations. Although the invention has been described in preferred forms with a certain degree of particularity, it is to be understood that the present disclosure has been made by way of example only.

Numerous changes in the details of the compositions and ingredients therein as well as in methods of preparation and use will be apparent to those skilled in the art without departing from the spirit and scope of the invention as defined in the appended claims.

### REFERENCES

[1] Untergasser A, Cutcutache I, Koressaar T, Ye J, Faircloth B C, Remm M, et al. Primer3—new capabilities and interfaces. Nucleic Acids Research. 2012; 40:e115-e.
[2] Ye J, Coulouris G, Zaretskaya I, Cutcutache I, Rozen S, Madden T L. Primer-BLAST: a tool to design target-specific primers for polymerase chain reaction. BMC Bioinformatics. 2012; 13:134.
[3] Dieffenbach C, Lowe T, Dveksler G. General concepts for PCR primer design. PCR Methods and Applications. 1993; 3:S30-S7.
[4] Hyndman D L, Mitsuhashi M. PCR primer design. PCR Protocols: Springer; 2003. p. 81-8.
[5] Mann T, Humbert R, Dorschner M, Stamatoyannopoulos J, Noble W S. A thermodynamic approach to PCR primer design. Nucleic Acids Research. 2009; 37:e95-e.
[6] Peters I R, Helps C R, Hall E J, Day M J. Real-time RT-PCR: considerations for efficient and sensitive assay design. Journal of Immunological Methods. 2004; 286:203-17.
[7] Rozen S, Skaletsky H. Primer3 on the WWW for general users and for biologist programmers. Bioinformatics Methods and Protocols: Springer; 1999. p. 365-86.
[8] Ginzinger D G. Gene quantification using real-time quantitative PCR: an emerging technology hits the mainstream. Experimental Hematology. 2002; 30:503-12.
[9] Kovárová M, Dráber P. New specificity and yield enhancer of polymerase chain reactions. Nucleic Acids Research. 2000; 28:e70-e.
[10] Lebedev A V, Paul N, Yee J, Timoshchuk V A, Shum J, Miyagi K, et al. Hot start PCR with heat-activatable primers: a novel approach for improved PCR performance. Nucleic Acids Research. 2008; 36:e131-e.
[11] Mikeska T, Dobrovic A. Validation of a primer optimisation matrix to improve the performance of reverse transcription-quantitative real-time PCR assays. BMC Research Notes. 2009; 2:112.
[12] Reynisson E, Josefsen M H, Krause M, Hoorfar J. Evaluation of probe chemistries and platforms to improve the detection limit of real-time PCR. Journal of Microbiological Methods. 2006; 66:206-16.
[13] Huggett J, Bustin S A. Standardisation and reporting for nucleic acid quantification. Accreditation and Quality Assurance. 2011; 16:399-405.
[14] Huggett J, Dheda K, Bustin S, Zumla A. Real-time RT-PCR normalisation; strategies and considerations. Genes & Immunity. 2005; 6:279-84.
[15] Ashlock D, Wittrock A, Wen T-J. Training finite state machines to improve PCR primer design. Computational Intelligence, Proceedings of the World on Congress on: IEEE; 2002. p. 13-8.
[16] Latorra D, Arar K, Hurley J M. Design considerations and effects of LNA in PCR primers. Molecular and Cellular Probes. 2003; 17:253-9.
[17] Tichopad A, Dzidic A, Pfaffl M W. Improving quantitative real-time RT-PCR reproducibility by boosting primer-linked amplification efficiency. Biotechnology Letters. 2002; 24:2053-6.
[18] Afonina I, Ankoudinova I, Mills A, Lokhov S, Huynh P, Mahoney W. Primers with 5′ flaps improve real-time PCR. BioTechniques. 2007; 43:770.
[19] Sachs A B. Messenger RNA degradation in eukaryotes. Cell. 1993; 74:413-21.
[20] Houseley J, Tollervey D. The many pathways of RNA degradation. Cell. 2009; 136:763-76.
[21] Frazão C, McVey C E, Amblar M, Barbas A, Vonrhein C, Arraiano C M, et al. Unravelling the dynamics of RNA degradation by ribonuclease II and its RNA-bound complex. Nature. 2006; 443:110-4.
[22] van Hoof A, Parker R. Messenger RNA degradation: beginning at the end. Current Biology. 2002; 12:R285-R7.
[23] Christodoulou D C, Gorham J M, Herman D S, Seidman J. Construction of normalized RNA-seq libraries for Next-Generation Sequencing using the crab duplex-specific nuclease. Current Protocols in Molecular Biology. 2011:4.12. 1-4 . . . 1.
[24] Fleige S, Waif V, Huch S, Prgomet C, Sehm J, Pfaffl M W. Comparison of relative mRNA quantification models and the impact of RNA integrity in quantitative real-time RT-PCR. Biotechnology Letters. 2006; 28:1601-13.
[25] Rowley J W, Oler A J, Tolley N D, Hunter B N, Low E N, Nix D A, et al. Genome-wide RNA-seq analysis of human and mouse platelet transcriptomes. Blood. 2011; 118:e101-e11.
[26] Schroeder A, Mueller O, Stocker S, Salowsky R, Leiber M, Gassmann M, et al. The RIN: an RNA integrity number for assigning integrity values to RNA measurements. BMC Molecular Biology. 2006; 7:3.
[27] Auer H, Lyianarachchi S, Newsom D, Klisovic M I. Chipping away at the chip bias:

RNA degradation in microarray analysis. Nature Genetics. 2003; 35:292-3.
[28] Fleige S, Pfaff! M W. RNA integrity and the effect on the real-time qRT-PCR performance. Molecular Aspects of Medicine. 2006; 27:126-39.
[29] Romero I G, Pai A A, Tung J, Gilad Y. RNA-seq: Impact of RNA degradation on transcript quantification. BMC Biology. 2014; 12:42.
[30] Antonov J, Goldstein D R, Oberli A, Baltzer A, Pirotta M, Fleischmann A, et al. Reliable gene expression measurements from degraded RNA by quantitative real-time PCR depend on short amplicons and a proper normalization. Laboratory Investigation. 2005; 85:1040-50.
[31] Fleming R I, Harbison S. The development of a mRNA multiplex RT-PCR assay for the definitive identification of body fluids. Forensic Science International: Genetics. 2010; 4:244-56.
[32] Haas C, Klesser B, Maake C, Bar W, Kratzer A. mRNA profiling for body fluid identification by reverse transcription endpoint PCR and realtime PCR. Forensic Science International: Genetics. 2009; 3:80-8.
[33] Hanson E K, Ballantyne J. Rapid and inexpensive body fluid identification by RNA profiling-based multiplex High Resolution Melt (HRM) analysis. F1000Research. 2013; 2:281.
[34] Juusola J, Ballantyne J. Multiplex mRNA profiling for the identification of body fluids. Forensic Science International. 2005; 152:1-12.
[35] Cox M P, Peterson D A, Biggs P J. SolexaQA: At-a-glance quality assessment of Illumine second-generation sequencing data. BMC Bioinformatics. 2010; 11:485.
[36] http://hgdownload.cse.ucsc.edu/goldenPath/hg19/database/. UCSC Genome Bioinformatics. 2014.
[37] Kim D, Pertea G, Trapnell C, Pimentel H, Kelley R, Salzberg S L. TopHat2: accurate alignment of transcriptomes in the presence of insertions, deletions and gene fusions. Genome Biology. 2013; 14:R36.
[38] Trapnell C, Roberts A, Goff L, Pertea G, Kim D, Kelley D R, et al. Differential gene and transcript expression analysis of RNA-seq experiments with TopHat and Cufflinks. Nature Protocols. 2012; 7:562-78.
[39] Trapnell C. Cuffmerge Documentation, v3 Print-icon [symbol] Open Module on GenePattern Public Server.
[40] Koressaar T, Remm M. Enhancements and modifications of primer design program Primer3. Bioinformatics. 2007; 23:1289-91.

What we claim is:

1. A method for the detection of an RNA sequence in a sample, the method including the steps:
a) providing a sample, and
b) detecting the RNA sequence using at least one primer or probe complementary to a stable region of the RNA sequence.

2. A method according to claim 1 wherein stable region of the RNA sequence has been identified using RNA sequencing of the sample.

3. A method according to claim 1 or claim 2 wherein the stable region of the RNA sequence has been identified as a region in the RNA sequence which has more aligned sequencing reads than another region, or regions, of the same RNA sequence.

4. A method according to any one of claims 1 to 3 wherein the stable region is selected from the group comprising SEQ ID NO:6 to SEQ ID NO:10 and SEQ ID NO:39 to SEQ ID NO:56 or a compliment of anyone thereof.

5. A method according to any one of claims 1 to 4, wherein the primer is selected from the group comprising SEQ ID NO:11 to SEQ ID NO:20 or compliment of anyone thereof.

6. A method according to any one of claims 1 to 4, wherein the probe is selected from the group comprising SED ID NO:57 to SEQ ID NO:92 or compliment of anyone thereof.

7. A method according to any one of claims 1 to 6, wherein the sample is a biological tissue sample.

8. A method according to claim 7, wherein the sample is a solid sample.

9. A method according to claim 7, wherein the sample is a liquid sample.

10. A method according to claim 7, wherein the sample is from an internal organ.

11. A method according to claim 7, wherein the sample is selected from the group comprising heart, brain, liver, fat, muscle, gastrointestinal tract, lung and bone.

12. A method according to any one of claims 1 to 6, wherein the sample is a forensic sample.

13. A method according to claim 12, wherein the forensic sample is selected from the group comprising blood, buccal, saliva, menstrual blood, skin, semen and vaginal fluid.

14. A method according to any one of claims 1 to 13, wherein RNA is extracted from the sample prior to the detecting step.

15. A method according to any one of claims 1 to 14, wherein the RNA sequence is detected directly.

16. A method according to any one of claims 1 to 14, wherein the RNA sequence is detected indirectly.

17. A method according to claim 16, wherein the RNA sequence is detected indirectly by detection of a complementary DNA (cDNA) corresponding to the RNA sequence.

18. A method of typing a sample including RNA, the method including the steps:
a) providing a sample including RNA;
b) detecting one or more stable RNA sequences in the sample using at least one primer or probe complementary to the one or more stable region of the RNA;
wherein the stable RNA sequence is specific for the type of sample; and
wherein detecting the stable RNA sequence indicates the type of sample.

19. A method according to claim 19 wherein stable region of the RNA sequence has been identified using RNA sequencing of the sample.

20. A method according to claim 18 or claim 19 wherein the stable region of the RNA sequence has been identified as a region in the RNA sequence which has more aligned sequencing reads than another region, or regions, of the same RNA sequence.

21. A method according to any one of claims 18 to 20 wherein the stable region is selected from the group comprising SEQ ID NO:6 to SEQ ID NO:10 and SEQ ID NO:39 to SEQ ID NO:56 or a compliment of anyone thereof.

22. A method according to any one of claims 18 to 21, wherein the primer is selected from the group comprising SEQ ID NO:11 to SEQ ID NO:20.

23. A method according to any one of claims 18 to 21, wherein the probe is selected from the group comprising SED ID NO:57 to SEQ ID NO:92 or compliment of anyone thereof.

24. A method according to any one of claims 18 to 23 wherein the sample is a biological tissue sample.

25. A method according to any one of claims 18 to 24, wherein the sample is a solid sample.

26. A method according to any one of claims 18 to 24, wherein the sample is a liquid sample.

27. A method according to any one of claims 18 to 26, wherein the sample is from an internal organ.

28. A method according to any one of claims 18 to 27, wherein the sample is selected from the group comprising heart, brain, liver, fat, muscle, gastrointestinal tract, lung and bone.

29. A method according to any one of claims 18 to 27, wherein the sample is a forensic sample.

30. A method according to claim 29, wherein the forensic sample is selected from the group comprising blood, buccal, saliva, menstrual blood, skin, semen and vaginal fluid.

31. A method according to any one of claims 18 to 30, wherein RNA is extracted from the sample prior to the detecting step.

32. A method according to any one of claims 18 to 31, wherein the RNA sequence is detected directly.

33. A method according to any one of claims 18 to 31, wherein the RNA sequence is detected indirectly.

34. A method according to claim 33, wherein the RNA sequence is detected indirectly by detection of a complementary DNA (cDNA) corresponding to the RNA sequence.

35. A method of typing a sample including degraded RNA, the method including the steps:
a) providing a sample including degraded RNA;
b) detecting one or more stable RNA sequences in the sample using at least one primer or probe complementary to the one or more stable region of the degraded RNA;
wherein the stable RNA sequence is specific for the type of sample; and
wherein detecting the target RNA sequence indicates the type of sample.

36. A method according to claim 35 wherein stable region of the RNA sequence has been identified using RNA sequencing of the sample.

37. A method according to claim 35 or claim 36 wherein the stable region of the RNA sequence has been identified as a region in the RNA sequence which has more aligned sequencing reads than another region, or regions, of the same RNA sequence.

38. A method according to any one of claims 35 to 37 wherein the stable region is selected from the group comprising SEQ ID NO:6 to SEQ ID NO:10 and SEQ ID NO:39 to SEQ ID NO:56 or a compliment of anyone thereof.

39. A method according to any one of claims 35 to 38, wherein the primer is selected from the group comprising SEQ ID NO:11 to SEQ ID NO:20.

40. A method according to any one of claims 35 to 38, wherein the probe is selected from the group comprising SED ID NO:57 to SEQ ID NO:92 or compliment of anyone thereof.

41. A method according to any one of claims 35 to 40 wherein the sample is a biological tissue sample.

42. A method according to any one of claims 35 to 41, wherein the sample is a solid sample.

43. A method according to any one of claims 35 to 41, wherein the sample is a liquid sample.

44. A method according to any one of claims 35 to 43, wherein the sample is from an internal organ.

45. A method according to any one of claims 35 to 43, wherein the sample is selected from the group comprising heart, brain, liver, fat, muscle, gastrointestinal tract, lung and bone.

46. A method according to any one of claims 35 to 45, wherein the sample is a forensic sample.

47. A method according to claim 46, wherein the forensic sample is selected from the group comprising blood, buccal, saliva, menstrual blood, skin, semen and vaginal fluid.

48. A method according to any one of claims 35 to 47, wherein RNA is extracted from the sample prior to the detecting step.

49. A method according to any one of claims 35 to 48, wherein the RNA sequence is detected directly.

50. A method according to any one of claims 35 to 48, wherein the RNA sequence is detected indirectly.

51. A method according to claim 50, wherein the RNA sequence is detected indirectly by detection of a complementary DNA (cDNA) corresponding to the RNA sequence.

52. A method for the identification of a stable region in RNA in a sample, the method comprising:
a) providing a sample including RNA,
b) isolating total RNA from the sample,
c) removing DNA from the sample
d) generating cDNA complementary to the RNA in the sample,
e) sequencing the cDNA
wherein the stable region of the RNA sequence is identified as a region in the RNA sequence which has more aligned sequencing reads than another region, or regions, of the same RNA sequence.

53. A method according to claim 52 wherein the RNA is degraded.

54. A method according to claim 52 or claim 53, wherein the stable region of the RNA sequence is identified as a region in the RNA sequence which has more aligned sequencing reads than another region, or regions, of the same RNA sequence.

59. A nucleotide sequence comprising at least 5 nucleotides with at least 70% identity to a sequence selected from SEQ ID NO:6 to SEQ ID NO:10 or a compliment thereof, or a sequence selected from SEQ ID NO:39 to SEQ ID NO:56 or a compliment thereof.

60. A nucleotide sequence comprising at least 5 nucleotides of a sequence selected from SEQ ID NO:6 to SEQ ID NO:10 or a compliment thereof, or a sequence selected from SEQ ID NO:39 to SEQ ID NO:56 or a compliment thereof.

61. A nucleotide sequence selected from any one of SEQ ID NO:11 to SEQ ID NO:20.

62. Use of a nucleotide sequence according to any one of claims 59 to 61 in the typing of a sample including RNA.

65. A microarray comprising a sequence of at least 5 nucleotides with at least 70% identity to any part of the sequence of any one of SEQ ID NO:6 to SEQ ID NO:10 or a complement thereof.

66. A microarray comprising a sequence of at least 5 nucleotides of a sequence of any one of SEQ ID NO:6 to SEQ ID NO:10 or a complement thereof.

67. A microarray comprising a sequence of at least 10 nucleotides of a sequence with at least 70% identify to any part of the sequence of any one of SEQ ID NO:6 to SEQ ID NO:10 or a complement thereof.

68. A microarray comprising a sequence of at least 10 nucleotides of a sequence of any one of SEQ ID NO: 6 to SEQ ID NO: 10 or a complement thereof.

69. A microarray comprising a sequence of at least 5 nucleotides with at least 70% identity to any part of the sequence of any one of SEQ ID NO: 39 to SEQ ID NO:56 or a complement thereof.

70. A microarray comprising a sequence of at least 5 nucleotides of a sequence of any one of SEQ ID NO: 39 to SEQ ID NO:56 or a complement thereof.

71. A microarray comprising a sequence of at least 10 nucleotides with at least 70% identity to any part of the sequence of any one of SEQ ID NO: 39 to SEQ ID NO:56 or a complement thereof.

72. A microarray comprising a sequence of at least 5 nucleotides with at least 70% identity to any part of the sequence of any one of SEQ ID NO:11 to SEQ ID NO:20 or a complement thereof.

73. A microarray comprising a sequence of at least 5 nucleotides of a sequence of any one of SEQ ID NO:11 to SEQ ID NO:20 or a complement thereof.

74. A microarray comprising a sequence of at least 10 nucleotides of a sequence with at least 70% identify to any part of the sequence of any one of SEQ ID NO:11 to SEQ ID NO:20 or a complement thereof.

75. A microarray comprising a sequence of at least 10 nucleotides of a sequence of any one of SEQ ID NO: 11 to SEQ ID NO: 20 or a complement thereof.

76. A microarray comprising a sequence of at least 5 nucleotides with at least 70% identity to any part of the sequence of any one of SEQ ID NO:57 to SEQ ID NO:92 or a complement thereof.

77. A microarray comprising a sequence of at least 5 nucleotides of a sequence of any one of SEQ ID NO:57 to SEQ ID NO:92 or a complement thereof.

78. A microarray comprising a sequence of at least 10 nucleotides of a sequence with at least 70% identify to any part of the sequence of any one of SEQ ID NO:57 to SEQ ID NO:92 or a complement thereof.

79. A microarray comprising a sequence of at least 10 nucleotides of a sequence of any one of SEQ ID NO:57 to SEQ ID NO:92 or a complement thereof.

80. A kit comprising a nucleotide sequence selected from SEQ ID NO:11 to SEQ ID NO:20, SEQ ID NO: 39 to SEQ ID NO:56, SEQ ID NO:57 to SEQ ID NO:92 or a compliment thereof.
